(19) United States
(12) Patent Application Publication
(10) Pub. No.: US 2014/0053859 A1
(43) Pub. Date: Feb. 27, 2014

(54) NAIL COATINGS AND METHODS THEREOF

(71) Applicant: CREATIVE NAIL DESIGN, INC., Vista, CA (US)

(72) Inventors: David Valia, Vista, CA (US); Jamie Ellis, Vista, CA (US)

(73) Assignee: Creative Nail Design, Inc., Vista, CA (US)

(21) Appl. No.: 13/827,483

(22) Filed: Mar. 14, 2013

Related U.S. Application Data
(60) Provisional application No. 61/692,096, filed on Aug. 22, 2012.

(51) Int. Cl. A61K 8/891; A61Q 3/02; A61K 8/81; A45D 29/11; A61K 8/73

(52) U.S. Cl. CPC A61K 8/891; A45D 29/11; A61K 8/731; A61K 8/8135; A61K 8/8152; A61Q 3/02
USPC 132/200; 424/61

(57) ABSTRACT

The present embodiments relate to a nail coating having a major advantage in that it enables a nail coating to adhere to a natural nail or uncoated nail for long wear periods without adhesion loss or other signs of breakdown of the coating. Nail topcoats with improved abrasion resistance are also disclosed.

### FIELD OF THE DISCLOSURE

Embodiments of the present disclosure relate to nail coatings.

### BACKGROUND OF THE DISCLOSURE

The nail plate (i.e., the natural nail) is primarily composed of keratin, a water-insoluble, fibrous protein that is a major structural component of skin, hair, wool, silk, feathers, scales, nails and hooves. While keratins can obviously differ greatly in their amino acid makeup, hard keratins may all be generally characterized as cross-linked polypeptides. Alpha-keratins such as nails and hooves may be further characterized by their relatively higher percentages of the amino acid cysteine. Typically, the alpha-helix coils of the polypeptides are cross-linked with disulphide bonds between adjacent cysteines. The resulting plate-like cells are cemented to each other with a sticky substance and held together by rivet-like structures called desmosomes. Many cell layers adhere to each other to form the nail plate, a structure that resembles a brick and mortar wall.

Conventional natural nail coatings may be classified into two categories: nail polishes (also known as lacquers, varnish or enamels) and artificial nails (also known as gels or acrylics). Nail enamels typically comprise various solid components which are dissolved and/or suspended in non-reactive solvents. Upon application and drying, the solids deposit on the nail surface as a clear, translucent or colored film. Typically, nail polishes are easily scratched and are easily removable with solvent, usually within one minute and if not removed as described, will chip or peel from the natural nail in one to five days.

Nail enamels coat the surface of the nail plate to provide a decorative finish with a characteristic glossy finish. Nail enamels conventionally comprise a film forming component, which is frequently nitrocellulose, cellulose acetate butyrate, or a combination of one or both of those cellulosics with a polyester or other polymeric compound. Most nail polishes are made of nitrocellulose dissolved in a solvent (e.g. butyl acetate or ethyl acetate) and either left clear or colored with various pigments. Typical components may include: film forming agents, resins and plasticizers, solvents, and coloring agents.

Application of nail coatings to the surface of the nail plate typically requires the surface of the nail plate to be treated. The surface treatment typically involves the use of a primer and/or roughening of the nail plate such as with the use of a file. This treatment process may cause damage to the nail plate, which is particularly problematic for individuals having thin nails.

Primers are adhesion promoters that improve adhesion by increasing interfacial compatibility between surfaces, e.g., the nail plate and an applied coating. For example, a coating of nail polish will resist chipping and peeling if a good primer is used. Primers are more compatible with the nail plate than the nail polish. Primers act as the “go-between” or “anchor”, to improve adhesion.

Primers are also frequently used with artificial nail enhancements since acrylic nail products normally have poor adhesion to nail plates. In general, nail plate primers can be thought of as double-sided sticky tape, joining the nail plate to the nail enhancement. The nail plate surface is made up of chemical groups possessing specific structures. Primer molecules must match the chemical and structural characteristics of the nail plate. More particularly, one end of the primer is reactive with methacrylate monomers in the nail coating. With these types of primers, physical abrasion of the nail plate is required to achieve proper levels of adhesion to the keratin substrate. Moreover, these primers are corrosive, and if used improperly they can cause damage to the nail plate and surrounding tissue. These primers can also cause discoloration of the nail enhancement and are a leading cause of nail product discoloration.

Throughout this description, including the foregoing description of related art, any and all publicly available documents described herein, including any and all U.S. patents, are specifically incorporated by reference herein in their entirety. The foregoing description of related art is not intended in any way as an admission that any of the documents described therein, including pending United States patent applications, are prior art to embodiments of the present disclosure. Moreover, the description herein of any disadvantages associated with the described products, methods, and/or apparatus, is not intended to limit the disclosed embodiments. Indeed, embodiments of the present disclosure may include certain features of the described products, methods, and/or apparatus without suffering from their described disadvantages.

### SUMMARY OF THE DISCLOSURE

The nail coatings of the present embodiments eliminate the need to surface prep the nail plate with primers, filing or other means that may cause damage or otherwise thin (i.e., remove material from) the nail plate.

According to some embodiments, there is provided a nail coating that includes at least one film-forming agent; at least one silicone resin; and solvent. In some embodiments, the at least one film-forming agent is a cellulosic resin.

According to some embodiments, there is provided a nail coating including at least one film-forming agent; at least one plasticizer; at least one silicone resin; and solvent.

According to some embodiments, there is provided a nail coating containing at least one film-forming agent; at least one silicone resin; at least one coloring agent; and solvent. According to some embodiments, there is provided a nail coating that includes at least one film-forming agent; at least one plasticizer; at least one silicone resin; at least one coloring agent; and solvent. In some embodiments, the at least one film-forming agent is a cellulosic resin.

In some embodiments, the silicon resin and film forming agent, such as a cellulosic resin, are combined in a ratio of silicon resin to the film forming agent from about 1:10 to about 1:1. In some embodiments, the silicon resin is a polyhedral oligomeric silsesquioxane. In some embodiments, the cellulosic resin is nitrocellulose. In some embodiments, the cellulosic resin is a cellulose ester. In some embodiments, the cellulosic resin is a cellulose acetate alkylate. In some embodiments, the cellulosic resin is a cellulose acetate alkylate selected from the group consisting of cellulose acetate butyrate, cellulose acetate propionate, and mixtures thereof. In some embodiments, the plasticizer is an alkyl citrate. In some embodiments, the plasticizer is acetyl tributyl citrate. In some embodiments, the plasticizer is an acetylated monoglyceride.

According to some embodiments, there is provided a nail topcoat comprising: at least one film-forming agent; at least one silicone resin; a component selected from at least one ethylenically unsaturated monomer, at least one urethane (meth)acrylate resin or a combination thereof; at least one photoinitiator; and solvent.

According to some embodiments, there is provided a nail topcoat containing at least one film-forming agent; at least one plasticizer; at least one silicone resin; at least one photoinitiator; solvent; and a component selected from at least one ethylenically unsaturated monomer, at least one urethane (meth)acrylate resin or a combination thereof. In some embodiments, the plasticizer is an alkyl citrate. In some embodiments, the plasticizer is acetyl tributyl citrate. In some embodiments, the plasticizer is an acetylated monoglyceride.

According to some embodiments, there is provided a nail topcoat that includes at least one film-forming agent; at least one plasticizer; at least one silicone resin a component selected from at least one ethylenically unsaturated monomer, at least one urethane (meth)acrylate resin or a combination thereof; at least one high-molecular weight (meth)acrylate polymer or copolymer; at least one photoinitiator; and solvent. In this and other embodiments, the (meth)acrylate polymer or copolymer can be a copolymer of an alkyl (meth)acrylate and a (meth)acrylic acid. In some embodiments, the (meth)acrylate polymer or copolymer is a copolymer of an alkyl methacrylate and methacrylic acid. In some embodiments, the alkyl methacrylate is methyl methacrylate or butyl methacrylate.

According to some embodiments, there is provided a nail topcoat that includes at least one film-forming agent; at least one silicone resin; at least one ethylenically unsaturated monomer; at least one high-molecular weight (meth)acrylate polymer or copolymer; at least one photoinitiator; and solvent.

According to some embodiments, there is provided a nail topcoat containing at least one film-forming agent; at least one silicone resin; at least one ethylenically unsaturated monomer; at least one urethane (meth)acrylate resin; and at least one photoinitiator; and solvent.

According to some embodiments, there is provided a nail topcoat that includes at least one film-forming agent; at least one silicone resin; at least one urethane (meth)acrylate resin; at least one photoinitiator; and solvent.

According to some embodiments, there is provided a nail topcoat including at least one film-forming agent; at least one silicone resin; at least one ethylenically unsaturated monomer; at least one photoinitiator; and solvent.

According to some embodiments, there is provided a nail topcoat containing at least one film-forming agent; at least one silicone resin; at least one urethane (meth)acrylate resin; at least one high-molecular weight (meth)acrylate polymer or copolymer; at least one photoinitiator; and solvent.

According to some embodiments, there is provided a nail topcoat that includes at least one film-forming agent; at least one silicone resin; at least one ethylenically unsaturated monomer; at least one urethane (meth)acrylate resin; at least one high-molecular weight (meth)acrylate polymer or copolymer; at least one photoinitiator; and solvent.

In some embodiments, the plasticizer can be an alkyl citrate, for example acetyl tributyl citrate, or an acetylated monoglyceride. In some embodiments, the (meth)acrylate polymer or copolymer can be a copolymer of an alkyl (meth)acrylate and a (meth)acrylic acid, for example a copolymer of an alkyl methacrylate, such as butyl methacrylate or methyl methacrylate, and methacrylic acid.

According to some embodiments, there is provided a nail coating system that includes comprising the nail coating of the present embodiments and a nail topcoat. According to some embodiments, there is provided a nail coating system that includes a nail coating and the nail topcoat of the present embodiments.

According to some embodiments, there is provided a multilayer nail coating system including at least the nail coating of the present embodiments and a nail topcoat. According to some embodiments, there is provided a multilayer nail coating system including at least a nail coating and the nail topcoat of the present embodiments.

According to some embodiments, there is provided a method of applying a nail coating to an uncoated nail, the method including the steps of: applying the nail coating of the present embodiments to the uncoated nail. In some embodiments, the method further includes applying a nail topcoat to the coated nail surface.

According to some embodiments, there is provided a method of applying a nail coating to a natural nail that includes the steps of: applying a nail coating of the present embodiments to the natural nail. In some embodiments, the method also includes applying a nail topcoat to the coated nail surface. In some embodiments, the natural nail is not roughened or otherwise treated in order to promote the adhesion of a nail coating prior to applying the nail coating. In some embodiments, the natural nail is not surface treated with a primer prior to applying the coating. In some embodiments, the natural nail is not surface treated with a file prior to applying the coating.

### DETAILED DESCRIPTION OF THE INVENTION

### Nail Coating

The present application describes nail coatings. As compared to conventional nail enamels, the nail coating of the present embodiments has a major advantage in that it enables the nail coating, which may also contain color, to adhere to the natural nail for long wear periods without adhesion loss or other signs of breakdown of the coating. The improved wear is achieved without the need of surface prepping the nail, such as with the use of primers or by slightly roughing the surface with a file or other means. For example, the nail coating of the present embodiments may be applied directly to the nail.

In some embodiments, it may be recommended to simply clean the surface of the nail to remove excess dirt and/or excess of natural oils. Cleaning of the nail surface may be achieved with the light use of solvent such as isopropyl alcohol or acetone.

According to some embodiments, nail coatings of the present invention include: at least one film-forming agent; at least one silicone resin; at least one coloring agent; and solvent. According to some embodiments, nail coatings of the present invention contain at least one film-forming agent, at least one plasticizer, at least one silicone resin; at least one coloring agent; and solvent.

According to some embodiments, there is provided a nail coating comprising: between about 1.5 weight % to about 35 weight % of at least one film-forming agent; between about 1 weight % to about 10 weight % of at least one silicone resin; and between about 50 weight % to about 70 weight % of solvent.

According to some embodiments, nail coatings of the present invention include between about 1.5 weight % to about 35 weight % of at least one film-forming agent; between about 0 weight % to about 10 weight % of at least one plasticizer; between about 1 weight % to about 10 weight % of at least one silicone resin; and between about 50 weight % to about 70 weight % of solvent. In embodiments, the nail coating can also include between about 0 weight % to about 10 weight % of at least one coloring agent.

In some embodiments, the nail coatings according to the present invention include between about 1.5 weight % to about 50 weight % of at least one film-forming agent. In some embodiments, the nail coating includes between about 10 weight % to about 50 weight % of at least one film-forming agent. In some embodiments, the nail coating contains between about 1.5 weight % to about 35 weight % of at least one film-forming agent. In some embodiments, the nail coating contains between about 8 weight % to about 20 weight % of at least one film-forming agent. In some embodiments, the nail coating contains between about 10 weight % to about 35 weight % of at least one film-forming agent. In some embodiments, the nail coating contains between about 10 weight % to about 25 weight % of at least one film-forming agent. In some embodiments, the nail coating includes between about 10 weight % to about 20 weight % of at least one film-forming agent. In some embodiments, the nail coating includes between about 20 weight % to about 35 weight % of at least one film-forming agent.

In some embodiments, nail coatings according to the present invention can contain between about 0 weight % to about 20 weight % of at least one plasticizer. In some embodiments, the nail coatings include between about 0.5 weight % to about 20 weight % of at least one plasticizer. In some embodiments, the nail coating includes between about 1 weight % to about 10 weight % of at least one plasticizer. In some embodiments, the nail coating includes between about 2 weight % to about 10 weight % of at least one plasticizer. In some embodiments, the nail coating includes between about 3 weight % to about 10 weight % of at least one plasticizer. In some embodiments, the nail coating includes between about 5 weight % to about 10 weight % of at least one plasticizer. In some embodiments, the nail coating includes between about 3 weight % to about 7 weight % of at least one plasticizer. In some embodiments, the plasticizer is present in the amount of at least 1 weight %. In some embodiments, the plasticizer is present in the amount of at least 3 weight %. In some embodiments, the nail coating includes between about 1 weight % to about 3 weight % of a plasticizer. In some embodiments, the nail coating includes between about 1 weight % to about 7 weight % of a plasticizer.

In some embodiments, the at least one plasticizer is an acetylated monoglyceride. In some embodiments, the acetylated monoglyceride is present in the amount of at least 1 weight %. In some embodiments, the acetylated monoglyceride is present in the amount of at least 3 weight %. In some embodiments, the nail coating includes between about 1 weight % to about 3 weight % of an acetylated monoglyceride. In some embodiments, the nail coating includes between about 1 weight % to about 7 weight % of an acetylated monoglyceride.

In some embodiments, the at least one plasticizer is an alkyl citrate. In some embodiments, the alkyl citrate is present in the amount of at least 1 weight %. In some embodiments, the alkyl citrate is present in the amount of at least 3 weight %. In some embodiments, the nail coating contains between about 1 weight % to about 3 weight % of an alkyl citrate. In some embodiments, the nail coating contains between about 1 weight % to about 7 weight % of an alkyl citrate. In some embodiments, the alkyl citrate is acetyl tributyl citrate.

In some embodiments, nail coatings according to the present invention can contain between about 1 weight % to about 10 weight % of at least one silicone resin. In some embodiments, the nail coating contains between about 3 weight % to about 10 weight % of at least one silicone resin. In some embodiments, the nail coating contains between about 5 weight % to about 10 weight % of at least one silicone resin. In some embodiments, the nail coating contains between about 3 weight % to about 7 weight % of at least one silicone resin. In some embodiments, the silicone resin is a polyhedral oligomeric silsesquioxane.

In some embodiments, nail coatings of the present invention include between about 1 weight % to about 10 weight % of at least one coloring agent. In some embodiments, the nail coating includes between about 3 weight % to about 10 weight % of at least one coloring agent. In some embodiments, the nail coating includes between about 5 weight % to about 10 weight % of at least one coloring agent. In some embodiments, the nail coating includes between about 3 weight % to about 7 weight % of at least one coloring agent. In some embodiments, the at least one coloring agent may be present in the nail coating composition in an amount up to about 5% by weight relative to the total weight of the composition. In some embodiments, the at least one coloring agent is present in an amount of between about 2% by weight to about 3% by weight.

In some embodiments, nail coatings of the present invention include between about 50 weight % to about 70 weight % solvent. In some embodiments, the nail coating includes between about 60 weight % to about 70 weight % solvent. In some embodiments, the nail coating includes between about 55 weight % to about 65 weight % solvent.

The nail coating of the present embodiments may be applied to the nail in conjunction with the use of a nail topcoat. For example, the nail coating, optionally containing a color agent, is applied to an uncoated or natural nail surface, allowed to dry, and then a nail topcoat is applied on top of the nail coating. The nail topcoat may be any topcoat known in the art. In some embodiments, the nail topcoat is a nail topcoat according to the present embodiments, described in more detail herein below.

### Nail Topcoat

According to some embodiments, there is also provided a novel nail topcoat having improved abrasion resistance. According to some embodiments, nail topcoats according to the invention include at least one film-forming agent, at least one silicone resin; at least one photoinitiator; a component selected from at least one ethylenically unsaturated monomer and, at least one urethane (meth)acrylate resin or a combination thereof; and solvent. According to some embodiments, there is provided a nail topcoat including at least one film-forming agent, at least one plasticizer, at least one silicone resin; a component selected from at least one ethylenically unsaturated monomer and, at least one urethane (meth)acrylate resin or a combination thereof; at least one photoinitiator; and solvent. According to some embodiments, there is provided a nail topcoat that includes at least one film-forming agent, at least one silicone resin; at least one ethylenically unsaturated monomer; at least one photoinitiator; and solvent. According to some embodiments, nail topcoats of the present invention can contain at least one film-forming agent, at least one silicone resin; at least one ethylenically unsaturated monomer; at least one high-molecular weight (meth)acrylate polymer or copolymer; at least one photoinitiator; and solvent. According to some embodiments, a nail topcoat contains at least one film-forming agent, at least one silicone resin; at least one ethylenically unsaturated monomer; at least one urethane (meth)acrylate; at least one photoinitiator; and solvent. According to some embodiments, there is provided a nail topcoat containing at least one film-forming agent; at least one silicone resin; at least one urethane (meth)acrylate resin; at least one photoinitiator; and solvent. According to some embodiments, there is provided a nail topcoat containing at least one film-forming agent; at least one silicone resin; at least one urethane (meth)acrylate resin; at least one high-molecular weight (meth)acrylate polymer or copolymer; at least one photoinitiator; and solvent. According to some embodiments, there is provided a nail topcoat containing at least one film-forming agent; at least one silicone resin; at least one ethylenically unsaturated monomer; at least one urethane (meth)acrylate resin; at least one high-molecular weight (meth)acrylate polymer or copolymer; at least one photoinitiator; and solvent.

According to some embodiments, nail topcoats of the invention contain between about 5 weight % to about 40 weight % of at least one film-forming agent; between about 0 weight % to about 10 weight % of at least one plasticizer; between about 0.1 weight % to about 10 weight % of at least one silicone resin; between about 50 weight % to about 70 weight % of solvent; between about 0.5 weight % to about 10 weight % of at least one ethylenically unsaturated monomer; between about 0.5 weight % to about 10 weight % of at least one high-molecular weight (meth)acrylate; and between about 0.1 weight % to about 5 weight % of at least one photoinitiator.

According to some embodiments, nail topcoats of the invention contain between about 5 weight % to about 40 weight % of at least one film-forming agent; between about 0 weight % to about 5 weight % of at least one plasticizer; between about 0.1 weight % to about 5 weight % of at least one silicone resin; between about 50 weight % to about 70 §weight % of solvent; between about 0.5 weight % to about 7 weight % of at least one ethylenically unsaturated monomer; between about 0.5 weight % to about 5 weight % of high-molecular weight (meth)acrylate; and between about 0.005 weight % to about 5 weight % of photoinitiator.

In some embodiments, the nail topcoat contains between about 1.5 weight % to about 50 weight % of at least one film-forming agent. In some embodiments, the nail topcoat contains between about 10 weight % to about 50 weight % of at least one film-forming agent. In some embodiments, the nail topcoat contains between about 1.5 weight % to about 35 weight % of at least one film-forming agent. In some embodiments, the nail topcoat contains between about 8 weight % to about 20 weight % of at least one film-forming agent. In some embodiments, the nail topcoat contains between about 10 weight % to about 35 weight % of at least one film-forming agent. In some embodiments, the nail topcoat contains between about 10 weight % to about 25 weight % of at least one film-forming agent. In some embodiments, the nail topcoat contains between about 10 weight % to about 20 weight % of at least one film-forming agent. In some embodiments, the nail topcoat contains between about 20 weight % to about 35 weight % of at least one film-forming agent.

In embodiments that include a plasticizer, nail topcoats according to the invention can contain between about 0.1 weight % to about 5 weight % of at least one plasticizer. In some embodiments, the nail topcoat contains about 0.5 weight % to about 20 weight % of at least one plasticizer. In some embodiments, the nail topcoat contains between about 1 weight % to about 10 weight % of at least one plasticizer. In some embodiments, the nail topcoat contains between about 2 weight % to about 10 weight % of at least one plasticizer. In some embodiments, the nail topcoat contains between about 3 weight % to about 10 weight % of at least one plasticizer. In some embodiments, the nail topcoat contains between about 5 weight % to about 10 weight % of at least one plasticizer. In some embodiments, the nail topcoat contains between about 3 weight % to about 7 weight % of at least one plasticizer. In some embodiments, the plasticizer is present in the amount of at least 1 weight %. In some embodiments, the plasticizer is present in the amount of at least 3 weight %. In some embodiments, the nail topcoat comprises between about 1 weight % to about 3 weight % of a plasticizer. In some embodiments, the nail topcoat contains between about 1 weight % to about 7 weight % of a plasticizer.

In some embodiments, nail topcoats according to the present invention contain between about 1 weight % to about 10 weight % of at least one silicone resin. In some embodiments, the nail topcoat contains between about 3 weight % to about 10 weight % of at least one silicone resin. In some embodiments, the nail topcoat contains between about 5 weight % to about 10 weight % of at least one silicone resin. In some embodiments, the nail topcoat contains between about 3 weight % to about 7 weight % of at least one silicone resin. In some embodiments, the silicone resin is a polyhedral oligomeric silsesquioxane.

In some embodiments, a nail topcoat according to the present invention can contain between about 0.005 weight % to about 5 weight % photoinitiator. In some embodiments, the nail topcoat contains between about 0.01 weight % to about 5 weight % photoinitiator. In some embodiments, the nail topcoat contains between about 0.1 weight % to about 5 weight % photoinitiator.

In some embodiments containing an ethylenically unsaturated monomer, the nail topcoat of the invention includes between about 0.5 weight % to about 10 weight % of at least one ethylenically unsaturated monomer. In some embodiments, the nail topcoat includes between about 0.5 weight % to about 7 weight % of at least one ethylenically unsaturated monomer.

In some embodiments containing a high molecular weight (meth)acrylate polymer or copolymer, the nail coating of the invention includes between about 0.5 weight % to about 10 weight % of the at least one (meth)acrylate polymer or copolymer. In some embodiments, the nail coating includes between about 0.5 weight % to about 5 weight % of at least one (meth)acrylate polymer or copolymer. In some embodiments, the nail coating includes between about 1 weight % to about 10 weight % of at least one (meth)acrylate polymer or copolymer. In some embodiments, the nail coating includes between about 3 weight % to about 10 weight % of at least one (meth)acrylate polymer or copolymer. In some embodiments, the nail coating includes between about 5 weight % to about 10 weight % of at least one (meth)acrylate polymer or copolymer. In some embodiments, the nail coating includes between about 3 weight % to about 7 weight % of at least one (meth)acrylate polymer or copolymer.

In some embodiments containing a urethane (meth)acrylate resin, the nail coating of the invention includes between about 0.5 weight % to about 10 weight % of at least one urethane (meth)acrylate resin. In some embodiments, the nail coating includes between about 0.5 weight % to about 5 weight % of at least one urethane (meth)acrylate resin. In some embodiments, the nail coating includes between about 1 weight % to about 10 weight % of at least one urethane (meth)acrylate resin. In some embodiments, the nail coating includes between about 3 weight % to about 10 weight % of at least one urethane (meth)acrylate resin. In some embodiments, the nail coating includes between about 5 weight % to about 10 weight % of at least one urethane (meth)acrylate resin. In some embodiments, the nail coating includes between about 3 weight % to about 7 weight % of at least one urethane (meth)acrylate resin.

In some embodiments, the nail topcoat of the invention contains between about 50 weight % to about 70 weight % solvent. In some embodiments, the nail topcoat contains between about 60 weight % to about 70 weight % solvent. In some embodiments, the nail topcoat contains between about 55 weight % to about 65 weight % solvent.

According to some embodiments, a nail topcoat according to the present invention can contain between about 1.5 weight % to about 40 weight % of at least one film-forming agent; between about 0.1 weight % to about 10 weight % of at least one plasticizer; between about 0.1 weight % to about 10 weight % of at least one silicone resin; between about 50 weight % to about 70 weight % of solvent; between about 0.5 weight % to about 10 weight % of at least one ethylenically unsaturated monomer; between about 0.5 weight % to about 10 weight % of at least one urethane (meth)acrylate resin; and between about 0.1 weight % to about 5 weight % of at least one photoinitiator.

According to some embodiments, nail topcoats according to the present invention can contain between about 5 weight % to about 40 weight % of at least one film-forming agent; between about 0.1 weight % to about 5 weight % of at least one plasticizer; between about 0.1 weight % to about 5 weight % of at least one silicone resin; between about 50 weight % to about 70 weight % of solvent; between about 0.5 weight % to about 7 weight % of at least one ethylenically unsaturated monomer; between about 0.5 weight % to about 5 weight % of urethane (meth)acrylate resin; and between about 0.1 weight % to about 5 weight % of photoinitiator.

### Multilayer System

According to some embodiments, there is provided a nail coating system that includes a nail coating according to the present embodiments and a nail topcoat. According to some embodiments, there is provided a multilayer nail coating system that includes at least a nail coating according to the present embodiments and a nail topcoat. The nail topcoat may be any topcoat known in the art. In some embodiments, the nail topcoat is a nail topcoat according to the present embodiments.

Nail coating systems of the present embodiments provide a nail coating to the nail having improved wear. “Wear,” as used herein, refers to the length of time the consistency, coverage, texture, and/or color of a material remains unnoticeably different when compared to the time of application, as viewed by the naked eye of a normal observer. Wear may be evaluated by a test involving the application of a human nail and evaluating the consistency, texture and color of the composition after an extended period of time. For example, the consistency, texture and/or color of a nail coating may be evaluated immediately following application and these characteristics may then be re-evaluated and compared after an individual has worn the nail coating for a certain amount of time, for example one day, five days, seven days, ten days, or longer. These characteristics may be evaluated with respect to other compositions, such as commercially available compositions, a control or standard.

The nail coatings of the present embodiments show long wear or improved wear as compared to enamel coatings know in the prior art. “Long wear” or “improved wear” refers to the ability of a nail coating to stay on for an extended period of time without damage such as imprinting, chipping or loss of adhesion. “Long wear” may also be described as the ability to retain the appearance of having been freshly or recently applied for an extended period of time, for example one day, five days, seven days, ten days, or longer. Such nail coatings can also be described as having good or effective staying power, in that they can resist transfer from the surface to which they are applied for an extended period of time, preferably under various conditions. “Long wear” is used interchangeably herein with “extended wear,” “increased wear,” or “longer wear.”

In some embodiments, nail coatings of the present embodiments are wearable for at least five days. In some embodiments, nail coatings of the present embodiments are wearable for at least seven days. In some embodiments, nail coatings of the present embodiments are wearable for at least ten days. In some embodiments, nail coatings of the present embodiments are wearable for at least seven to ten days. In some embodiments, nail coatings of the present embodiments are wearable for at least five to seven days.

### Film-Forming Agents

In some embodiments, the at least one film-forming agent in the coating (nail coating or nail topcoat) is a film-forming polymer. Film-forming polymers include polyesters; resins, such as polyurethane resins, alkyd resins, and polyvinyl resins such as polyvinyl acetate, polyvinyl chloride, polyvinylbutyrate; (meth)acrylic and vinyl copolymers such as styrene/butadiene copolymers, acrylate/vinyl acetate copolymers, acrylonitrile/butadiene copolymers, ethylene/vinyl acetate copolymers, and silicone resins other than POSS resins as defined herein. In some embodiments, the at least one film-forming agent is a cellulosic resin.

### Cellulosic Resins as Film-Forming Agents

According to some embodiments, the at least one film-forming agent is at least one cellulosic resin. In some embodiments, the cellulosic resin is the major film former in the enamel.

In some embodiments, the silicon resin and cellulosic resin are combined in a ratio of silicon resin to cellulosic resin from about 1:10 to about 1:1. This includes from about 1:10 to about 1:4, from about 1:10 to about 1:6, from about 1:10 to about 1:8, from about 1:2 to about 1:1, from about 1:4 to about 1:1, from about 1:6 to about 1:1, from about 1:8 to about 1:1; from about 1:8 to about 1:4, from about 1:6 to about 1:4, from about 1:6 to about 1:2, and from about 1:8 to about 1:2.

In some embodiments, the cellulosic resin is nitrocellulose or other cellulose derivative, such as a cellulose ester, cellulose acetate alkylate (e.g., cellulose acetate propionate, cellulose acetate butyrate) and ethyl cellulose.

Nitrocellulose and cellulose esters useful in accordance with the present invention are identified in U.S. Pat. No. 6,333,025, the text of which is hereby incorporated by reference.

In some embodiments, the cellulosic resin is a nitrocellulose. Nitrocellulose can be present in the nail coating composition in an amount ranging from 1.5 to 35% by weight relative to the total weight of the composition. In some embodiments, the nitrocellulose can be present in the composition in an amount ranging from 8% to 20% by weight, relative to the total weight of the composition.

In some embodiments of the invention, the nail coatings may also include an additional film-forming agent, in an amount up to 50% by weight, and is preferably present in an amount less than 40% by weight, relative to the total weight of cellulose resin. In still another embodiment of the invention, the amount of additional film forming agent ranges from 1% to 15% by weight relative to the total weight of cellulose resin. These film-forming agents include polymers such as polyesters; resins, such as polyurethane resins, alkyd resins, and polyvinyl resins such as polyvinyl acetate, polyvinyl chloride, polyvinylbutyrate; (meth)acrylic and vinyl copolymers such as styrene/butadiene copolymers, acrylate/vinyl acetate copolymers, acrylonitrile/butadiene copolymers, and ethylene/vinyl acetate copolymers.

In some embodiments, the cellulosic resin is a cellulose ester. In some embodiments the cellulose ester is a cellulose acetate alkylate. The cellulose acetate alkylate may be selected from the group consisting of cellulose acetate butyrate, cellulose acetate propionate, and mixtures thereof.

### Silicone Resins

“Silicone resins” refers to a variety of polymers which are characterized by repeating Si subunits having at least one and up to four oxygen bridges with other Si atoms. Of the four possible Si bonds, instead of oxygen bridges, up to three R-groups can be present. By varying the subunits and substituents, a vast variety of polymers can be created. Silicone resins have been disclosed previously in U.S. Pat. No. 8,080,257, in which the silicone resins are used as a film-forming agents in conjunction with a liquid fatty phase that includes at least one hydrocarbon based polymer that includes a hetero atom as part of the polymer skeleton.

In some embodiments, the silicone resin may be either a siloxysilicate or a polysiloxane. Siloxysilicates have the formula [R₃—Si—O]ₓ—(SiO_{4/2})_{y}, wherein x and y range from about 50 to about 80, and polysiloxanes have the formula [R₃—Si—O]—(R₂SiO)_{X}—[Si—R₃], wherein X is at least 2000. The R groups can be, for example, an alkyl, hydroxyl, alkoxysilane, amine, chlorosilane, epoxide, ester, halide, methacrylate, molecular silica, nitrile, norbornene, olefin, phosphine, silane, silanol, styrenic polymer, or polyolefin.

In yet another instance, the siloxysilicate is of the formula [R₃—Si—O]ₓ—(SiO_{4/2})_{y}, wherein x and y range from about 50 to about 80, and R is an alkyl, hydroxyl, alkoxysilane, amine, chlorosilane, epoxide, ester, halide, methacrylate, molecular silica, nitrile, norbornene, olefin, phosphine, silane, silanol, styrenic polymer, or polyolefin.

In another instance, the siloxysilicate is a trimethylsiloxysilicate.

In some embodiments, the polysiloxane is of the formula [R₃—Si—O]—(R₂SiO)_{X}—[Si—R₃], wherein X is at least 2000, and R is an alkyl, hydroxyl, alkoxysilane, amine, chlorosilane, epoxide, ester, halide, methacrylate, molecular silica, nitrile, norbornene, olefin, phosphine, silane, silanol, styrenic polymer, or polyolefin. In some embodiments, the polysiloxane is dimethicone.

Among the silicone resins disclosed are polymethyl silsesquioxanes being formed primarily of polymerized repeating subunits of CH₃SiO_{3/2}. See also U.S. Pat. App. Pub. No. 2002/0031488, U.S. Pat. App. Pub. No. 2008/0081022, U.S. Pat. App. Pub. No. 2004/0202623, U.S. Pat. App. Pub. No. 2004/0202622, and U.S. Pat. No. 2,465,188, U.S. Pat. No. 5,047,492, U.S. Pat. No. 5,246,694, U.S. Pat. No. 5,439,673, U.S. Pat. No. 7,572,872, U.S. Pat. No. 7,226,960 and EP 0624594, the entire disclosures of which are incorporated herein by reference in their entireties.

Silicone resins are named in accordance with what is referred to in the art as “MDTQ” nomenclature, whereby a silicone resin is described depending upon the various monomeric siloxane subunits (“Si subunits”) which form the polymer. Each letter, “M,” “D,” “T” and “Q” stands for a different subunit. “M” denotes the monofunctional unit (CH₃)₃SiO_{1/2}. This unit is referred to as monofunctional because the silicone atom shares only one oxygen with another Si atom in the chain. The “M” unit can be represented by the structure:

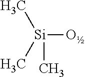

At least one of the methyl groups can be replaced, as demonstrated by the formula R(CH₃)₂SiO_{1/2}, where R can be a substituent other than a methyl group, for example a functional group or a longer alkyl group that may include functional groups as represented by the structure:

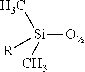

One or more of the methyl groups can be replaced by an R group which may be the same or different.

The letter “D” denotes the difunctional subunit (CH₃)₂SiO_{2/2} where two of the available bonds from the silicone atom are bound to oxygen in the formation of the polymeric chain. The “D” subunit can be represented as:

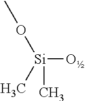

As is the case in connection with the M subunit, one or more methyl groups may be replaced with the same or different R groups as defined above.

The symbol “T” denotes the trifunctional subunit, (CH₃)SiO_{3/2} and can be represented as:

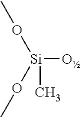

As illustrated above in connection with the “M” subunit, a methyl group can be replaced in the “T” subunits with another R group as defined above.

Finally, the symbol “Q” denotes the quadrifunctional subunit SiO_{4/2} which can be represented as:

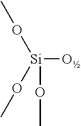

From this description, it is apparent that by varying the number and types of subunits, “M”, “D”, “T” and “Q”, and by varying the methyl or R groups, a vast number of combinations can be created.

In some embodiments, the silicone resin is a Q resin. By the term Q resin, it is meant that the resin contains predominantly Si subunits of the Q type, or that those of skill in the art would regard the particular resin predominantly as a Q resin. MQ resins are also referred to as “siloxysilicates”, such as trimethylsiloxysilicates, represented by the following formula: [(CH₃)₃—Si—O]ₓ—(SiO_{4/2})_{y} (MQ Units) where x and y can have values ranging from 50 to 80. In a further embodiment, the silicone resin is a siloxysilicate chosen from any combination of M and Q units, for example, [(R)₃—Si—O]ₓ—(SiO_{4/2})_{y}, wherein x and y can have values ranging from 50 to 80 and at least one R group is chosen from an alkyl group other than a methyl group, for example functional group or a longer hydrocarbon chain that may be functionalized. For example, a Q resin can be chosen from among the Wacker 803 and 804 resins, available from Wacker Silicone Corporation, and G.E. 1,170-002, available from General Electric.

The term “silsesquioxane” generally refers to a class of silicone resins of the T type (“T resins”). In some embodiments, the silicone resin can be chosen from silsesquioxanes represented by the following formula: (CH₃SiO_{3/2})ₓ (T Units) where x has a value of up to several thousand and the methyl may be replaced by another R group as described above for the M subunits. Note, however, that where a polymethylsilsesquioxane is employed, it is not combined with a POSS (Polyhedral Oligomeric Silsesquioxane) of the type having only 8 fully saturated Si subunits (complete cage of R₁ to R₈ methyl groups, as defined further below). Polymethylsilsesquioxanes are those silsesquioxanes wherein each substituent (R group) is a methyl group.

In some embodiments, the silicone resin is a “polyalkylsiloxane” or D resin. Again, by the term “D resin” it is meant that the resin contains predominantly Si subunits of the D type, or that those of skill in the art would regard the particular resin predominantly as a D resin. In some embodiments, the polysiloxane is of the formula [R₃—Si—O]—(R₂SiO)_{X}—[Si—R₃], wherein X is at least 2000, and R is an alkyl, hydroxyl, alkoxysilane, amine, chlorosilane, epoxide, ester, halide, methacrylate, molecular silica, nitrile, norbornene, olefin, phosphine, silane, silanol, styrenic polymer, or polyolefin. D resins include dimethylsiloxanes having the CTFA designation dimethicone. These siloxanes are available commercially from the General Electric Company as the Viscasil Series and from Dow Corning as the DC200 series.

### Substituted Silicone Resins

As stated previously, the M, D and T subunits may include one or more substituents (R-groups). As non-limiting examples, these R groups may include an alkyl, hydroxyl, alkoxysilane, amine, chlorosilane, epoxide, ester, halide, methacrylate, molecular silica, nitrile, norbornene, olefin, phosphine, silane, silanol, styrenic polymer or polyolefin.

More than one substitution can be made, wherein one, two or more of the methyl groups available are replaced with the same or different R groups. These groups can be directly bonded to the Si atom, or may be bound through a bridging moiety that may contain other functional groups, such as an azo, diazo, epoxy or halogen, which may be reactive functional groups.

### Polyhedral Oligomeric Silsesquioxanes or “POSS”

In some embodiments, the silicone resin is a Polyhedral Oligomeric (or Oligo) Silsequioxane (POSS). In some embodiments, the silicone resin is an extended Polyhedral Oligomeric (or Oligo) Silsequioxane (EPOSS) molecule containing six or more Si atoms within its cage-like structure. These compounds are distinguished from other silicone resins by their rigid three-dimensional cage-like structures. In some embodiments, the POSS used in the present embodiments has a three dimensional cage structure formed of a plurality of Si subunits, i.e. Si—O subunits, at least one of the subunits having one or more R groups. In some embodiments, the term “POSS” may refer to POSS molecules having 8 Si atoms or less (e.g., 6, 7 or 8), while EPOSS includes those cage structures having greater than 8 Si atoms. All silicone resins forming the cage structure may be used in the present embodiments. Accordingly, unless indicated otherwise, the term “POSS” refers to POSS or EPOSS molecules regardless of the number of Si atoms.

POSS are inorganic materials with a silica core and reactive functional groups on the surface and represented by the general formula of RSiO_{1.5}. Generally POSS are nano-sized, but may be larger depending upon the number of Si and O atoms in the structure, as well as substituents that might be present as described elsewhere herein. Cubic silsesquioxanes, such as octa(dimethylsiloxy)silsequioxane (R₈Si₈O₁₂), consist of a rigid, crystalline silica-like core that is well-defined spatially (0.5-0.7 nm) which can be linked covalently to eight R groups. A description of possible cages is discussed in U.S. Pat. No. 5,942,638. Each of the cages can be further modified by attaching reactive moieties to the cage atoms. The core accounts for approximately 5% of the total volume and the highly enhanced surface effects. By varying the functionality of the R group, it is possible to create octa-functional macromonomers that will self-polymerize or copolymerize with other functionalized cubes to provide nanocomposites whose length scales and interfacial interactions are well-defined. The structure of the organic phase between the rigid, hard particles can be varied systematically; the potential exists to carefully tune mechanical, optical properties to establish structure-property relationships.

In some embodiments, POSS refers to only those compounds existing in a rigid, “cage”-type configuration, examples of which are shown in Formulas I-V, below. In some embodiments, POSS refers to only certain structures, such as, by way of non-limiting examples, those illustrated in Formulas I, III and IVA, which are referred to herein as being “complete cages” wherein all of the sides of the three-dimensional structure are completed sides and all of the Si atoms are completely saturated.

In some embodiments, the coatings (nail coating or nail topcoat) of the present disclosure do not include other POSS that can exist, for example, in the ladder configuration of Formula VI. For example, the polymethylsilsesquioxane known as Resin MK, has previously been disclosed in connection with cosmetic formulations in U.S. Pat. App. Pub. No. US2002/0114773. As disclosed therein, the belief is that the compounds exist in both a “cage” (i.e., Formula I, wherein R₁-R₈ are CH₃—) and “ladder” configuration (Formula VI). It is also believed that the majority of the silicone polymers are present in the “ladder” configuration (Formula VI). To the extent that this composition contains the “ladder” configuration, it is not POSS as that term is used with respect to the present invention.

The POSS used in the nail coatings of the present embodiments may form the three-dimensional cage structure. In some embodiments, the POSS has at least 6 Si molecules. In some embodiments, the POSS contains 8 Si atoms. POSS may also include greater than 8 Si atoms or in mixtures containing, for example, 6-12 Si atoms or 8-12 Si atoms, for example as a mixture of compounds containing 8, 10 and 12 Si atoms. The number of Si atoms can also range from 6 to 100, alternatively 6 to 30, also alternatively 6 to 20 and finally alternatively 6 to 16, either as a single POSS structure (i.e. having the same configuration of Si and O atoms even if other substituents vary) or as a mixture of compounds with varying numbers of Si atoms with the same or varying R groups. In some embodiments, at least 4 of the Si atoms are bound, through an oxygen atom, to at least 3 other Si atoms (referred to herein as being “completely saturated”). All of the Si atoms are bound to at least one other Si atom through an oxygen bridge.

As shown in the exemplary and non-limiting structures of Formulas I through V and VII through X, POSS forms a rigid three-dimensional cage structure having at least two completed sides. This rigid cage structure is distinguished from ladders and other structures which are not held in place in three directions (See Formula VI for an exemplary ladder structure). Each of the Si atoms is bound to at least 1 R group with no more than 3, no more than 2 or no more than 1 Si atom being bound to more than 2 R groups. For example, the POSS molecule illustrated by Formula III has 6 saturated Si atoms and 5 complete sides (2 sides bounded by 3 Si atoms connected through oxygen bridges and 3 sides bounded by 4 Si atoms connected through oxygen bridges). Formula IIB has 4 such saturated Si atoms and 2 completed sides, (both bounded by 4 Si atoms connected through oxygen bridges). Formula IIC has 6 saturated Si atoms and 3 completed sides all bounded by 4 Si atoms connected through oxygen bridges.

POSS molecules in accordance with some embodiments have the complete cage structure of Formula I:

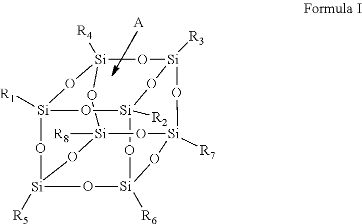

It is also possible that one or even two of the oxygen bridges between successive Si atoms are broken or missing, in which case the “POSS” is referred to as having an “incomplete” case structure. By way of non-limiting examples, consider the rigid three-dimensional cage structures illustrated in Formulas IIA-E:

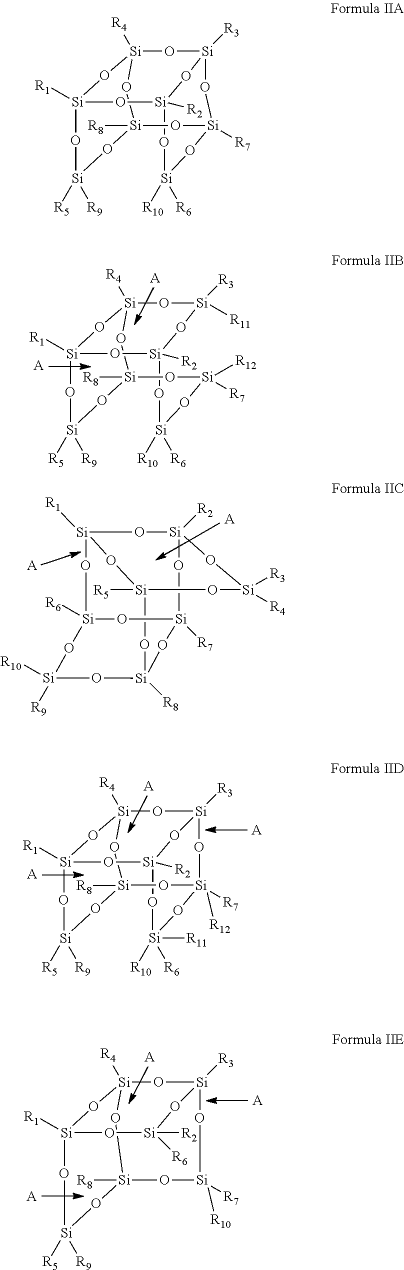

Formula III is a complete cage, but produced from 6 Si atoms.

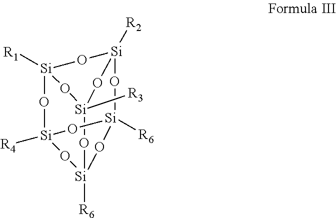

In Formula IVA, the number of Si atoms in the cage is 10, in Formula IVB, the number of Si atoms is 10 and in Formula IVC, the number of Si atoms in the cage is 12. In Formula IVD and IVE, the number of Si atoms in the cage or core is 16.

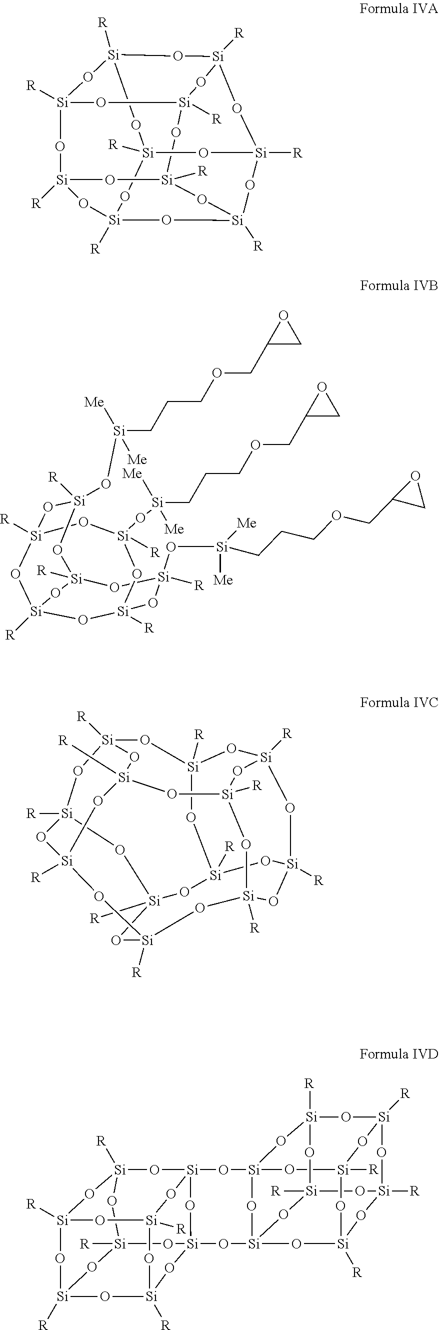

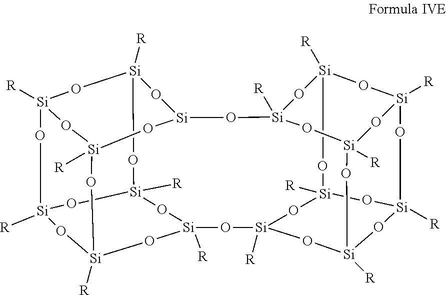

An example of an “incomplete” cage structure, wherein one or more of the oxygen bridges between successive Si atoms is broken or missing, is illustrated in Formula V:

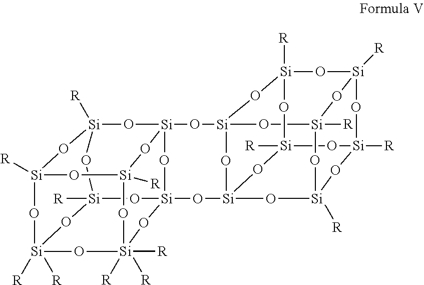

Formula VI, a ladder configuration (not a POSS according to the present embodiments/disclosure), can be a monomer linked end to end to other similar structures. It is not rigid within the meaning of this document as it can fold or flex around each R—Si—O—Si—R axis of the molecule. No such movement is possible in the rigid 3-D cage structures (whether complete or incomplete) of the POSS of the present embodiments. Thus, the molecules of this formula are not POSS.

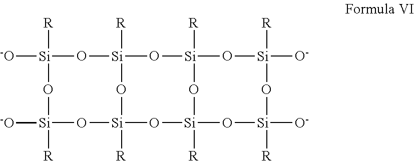

Note also that when referencing POSS molecules as being in accordance with Formula II, having the structure of Formula II, or being other than a completed cage, the sides which are illustrated as “open,” “missing” or “broken” are illustrative only. When reference is made to Formula II, it is understood that any one or two sides, or any one or two oxygen bridges, may be broken or missing. The structure of the POSS molecule can be roughly thought of as a box (prism in the case of Formula III) or cage in shape with silicon (Si) atoms at each corner. Each Si atom is connected to at least one other Si atom through bonds to an oxygen atom (also referred to as an “oxygen bridge”). Preferably, at least four of the Si atoms in the POSS structure are “completely saturated.” As used herein, an Si atom is “completely saturated” if bound, through oxygen atoms, to three other Si atoms within the cage as shown in Formulas I, III and IVA, most preferably, all of the Si atoms are “completely saturated”. While illustrated in Formula I as Si atoms, the groups at each corner may be the same or different and may be one or more atoms or groups including, without limitation, silicon, silane, siloxane, silicone or organometallic groups. The POSS of the invention also exists in a rigid 3-dimensional cage structure as illustrated, for example, in Formulas I-V and VII-X and the cage has at least two completed sides A. Each Si is bound to at least one R group. In some embodiments no more than one Si atom is bound to more than two R groups. In some embodiments no more than two Si atoms are bound to more than two R groups. In some embodiments no more than three Si atoms are bound to more than two R groups.

In some embodiments, polysilsesquioxanes are materials represented by the formula [RSiO_{1.5}]∞ where ∞ represents molar degree of polymerization and R=represents organic substituent (H, siloxy, cyclic or linear aliphatic or aromatic groups that may additionally contain reactive functionalities such as alcohols, esters, amines, ketones, olefins, ethers or halides or which may contain fluorinated groups). Polysilsesquioxanes may be either homoleptic or heteroleptic. Homoleptic systems contain only one type of R group while heteroleptic systems contain more than one type of R group.

Nanostructured chemicals are best exemplified by those based on low-cost Polyhedral Oligomeric Silsesquioxanes (POSS) and Polyhedral Oligomeric Silicates (POS). POSS systems contain hybrid (i.e., organic-inorganic) compositions in which the internal cage like framework is primarily comprised of inorganic silicon-oxygen bonds. The exterior of the nanostructure is covered by both reactive and nonreactive organic functionalities (R), which ensure compatibility and tailorability of the nanostructure with organic monomers and polymers.

POSS and POS nanostructure compositions are represented by the formulas:

[(RSiO_{1.5})ₙ]_{Σ#} for homoleptic compositions,

[(RSiO_{1.5})ₙ(R′SiO_{1.5})ₘ]_{Σ#} for heteroleptic compositions (where R≠R′),

[(RSiO_{1.5})ₙ(XSiO_{1.5})ₘ]_{Σ#} for functionalized heteroleptic compositions having a closed cage structure (where R groups can be equivalent or inequivalent). A functionalized heteroleptic POSS composition having an open cage structure may be represented by the formula [(RSiO_{1.5})ₙ(RXSiO_{1.0})ₘ]_{Σ#}.

By way of example, homoleptic POSS of Formulas III, I, IVA and IVC are designated as [(RSiO_{1.5})₆]Σ6, [(RSiO_{1.5})₆]Σ6, [(RSiO_{1.5})₈]Σ8, [(RSiO_{1.5})₁₀]Σ10 and [(RSiO_{1.5})₁₂]Σ12, respectively. Similarly, functionalized heteroleptic open cage POSS can have the following structures and designations:

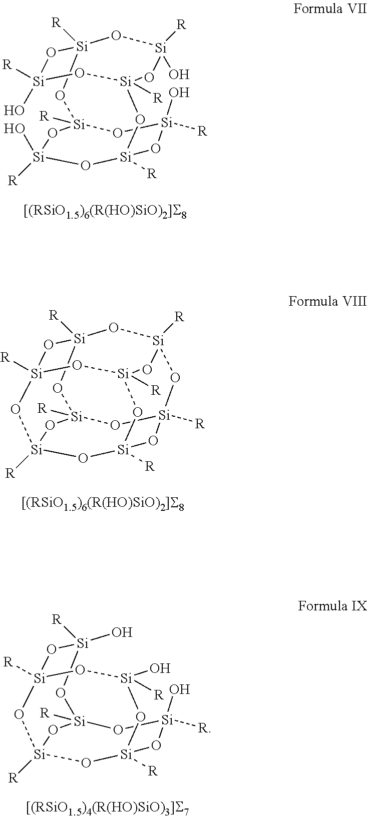

In all of the above structures and formulas, R is the same or different and can be any of the moieties as defined elsewhere herein and X includes but is not limited to OH, Cl, Br, I, alkoxide (OR), acetate (OCOR), acid (OCOH), ester (OCOR), peroxide (OOR), amine (NR₂), isocyanate (NCO), epoxy, olefin and R. The symbols m and n refer to the stoichiometry of the composition. The symbol Σ indicates that the composition forms a nanostructure and the symbol # refers to the number of silicon atoms contained within the nanostructure. The value for # is usually the sum of m+n, where n ranges typically from 1 to 24 and m ranges typically from 1 to 12. It should be noted that Σ# is not to be confused as a multiplier for determining stoichiometry, as it merely describes the overall nanostructural characteristics of the system (aka cage size).

Examples of attributes that enable nanostructured chemicals to function as 1-10 nm reinforcing agents include: (1) their unique size with respect to polymer chain dimensions, and (2) their ability to be compatibilized with polymer systems to overcome repulsive forces that promote incompatibility and expulsion of the nanoreinforcing agent by the polymer chains. That is, nanostructured chemicals can be tailored to exhibit preferential affinity/compatibility with some polymer microstructures through variation of the R groups on each nanostructure. At the same time, the nanostructured chemicals can be tailored to be incompatible or compatible with other microstructures within the same polymer, thus allowing for selective reinforcement of specific polymer microstructure. Therefore, the factors to effect a selective nanoreinforcement include specific nanosizes of nanostructured chemicals, distributions of nanosizes, and compatibilities and disparities between the nanostrucutured chemical and the polymer system. For POSS, dispersion of the molecules and their compatibility with polymer segments is thermodynamically governed by the free energy of mixing equation (ΔG=ΔH−TΔS). The nature of the R group and ability of the reactive groups on the POSS cage to react or interact with polymers and surfaces greatly contributes to a favorable enthalpic (ΔH) term while the entropic term (ΔS) for POSS is highly favorable because of the monoscopic cage size and distribution of 1.0.

### Substituted POSS

Similar to other silicone resins, the M, D or T subunits of a POSS can be “derivatized” by the replacement of a methyl or R group with a functional group other than a methyl or with a different R group. As non-limiting examples, one or more methyl or R groups could be replaced with another alkyl group, alkene, alkyne, hydroxyl, thiol, ester, acid, ether. In some embodiments, the “R groups” include, without limitation, one or more of the following: hydrogen, methyl, ethyl, propyl, isobutyl, isooctyl, phenyl, cyclohexyl, cyclopentyl, —OSi(CH₃)₂—CH₂—CH₂—(CF₂)₅CF₃, —(CH₂)₃SH, N⁺(CH₃)₃, O⁻N⁺CH₃)₃, —OH, —(CH₂)ₙN⁺H₃X⁻ wherein n is 0-30 and X is a counter ion,

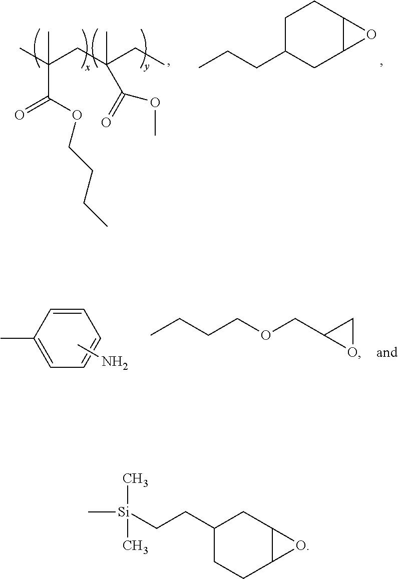

In some embodiments, R can also be a silane or siloxane structure, including a ladder structure. Formula X is a non-limiting example of a siloxane substituted POSS:

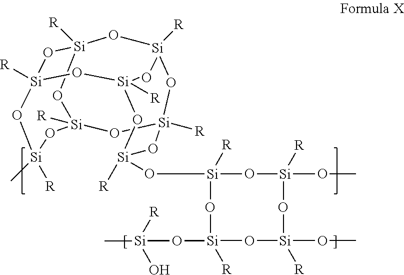

As previously illustrated (See Formulas IVD and IV E, the substituent can be an additional caged structure. In these instances, the structure can be considered conceptually as either a single POSS structure, as identified above, or as a POSS structure substituted by another POSS structure.

For example, the one remaining bond of each silicon of Formula I, III and IVA can bind to a variety of substituents or groups specified, as “R” groups (R₁-R₈), ((R₁-R₆) in Formula III). As used herein, when multiple R groups are present on the same POSS molecule, each R group may be the same or different whether all are designated as simply R or differentiated as R₁, R₂, R₃, . . . Rₙ. In some embodiments illustrated in Formulas II, IVB and V a POSS molecule in which one or two of the oxygen bridges between adjacent silicon molecules have been eliminated, a greater number of R groups are possible. When a POSS having 8 Si atoms is employed, it is preferred that no more than two of these inter-silicon connections (oxygen bridges) be eliminated. However, it is possible to eliminate as many as three such bridges (Formula IIE). More preferably, only a single oxygen bridge would be eliminated (Formula IIA). As stated above, the Si molecules not completely bound may have one or more additional positions available for binding additional substituents. In the case of a single missing side, the POSS molecule may include additional R groups R₉ and R₁₀, which may be the same or different as the R₁-R₈. When 2 or 3 bridges are missing, the POSS molecule may include additional R groups R₉, R₁₀, R₁₁ and R₁₂ (as appropriate), which all may be the same or different and may be the same as the groups identified for R₁-R₈.

POSS compounds with various R groups are known in the literature. They are described in a number of patents including, without limitation, U.S. Pat. No. 5,047,492; U.S. Pat. No. 5,389,726; U.S. Pat. No. 5,484,867; U.S. Pat. No. 5,589,562; U.S. Pat. No. 5,750,741; U.S. Pat. No. 5,858,544; U.S. Pat. No. 5,939,576; U.S. Pat. No. 5,942,638; U.S. Pat. No. 6,100,417; U.S. Pat. No. 6,127,557;U.S. Pat. No. 6,207,364; U.S. Pat. No. 6,252,030; U.S. Pat. No. 6,270,561; U.S. Pat. No. 6,277,451; U.S. Pat. No. 6,362,279; and U.S. Pat. No. 6,486,254. These patents describe in detail various methods of producing the basic POSS cage structure and various derivatives thereof, including POSS based polymers. To the extent that these patents identify and describe various POSS molecules having the structures of Formulas I-V and VII-X, derivatives and polymers thereof, they are incorporated by reference. The discussions of techniques for manufacturing and derivatizing this class of compounds described in each of these patents is also hereby incorporated by reference.

In general, R groups (for example, R₁, R₂, R₃, R₄, R₅, R₆, R₇, R₈, R₉, R₁₀, R₁₁ and R₁₂ as shown in the figures and any other R groups appropriate) can be the same or different and may be reactive or nonreactive groups. They may be, in replacing a methyl or H, for example, hydroxy (—OH), alkane derivatives (missing a hydrogen) also known as alkyl groups (other than methyl), alkenyl groups also referred to as derivatives of alkenes (having one or more double bonds), usually missing an H where they are bound to Si in POSS or to some other molecule, alkynyl groups also referred to as derivatives of alkynes (having one or more triple bonds) usually missing an H where they are bound to Si in POSS or to some other molecule, aryl groups (either the 6-carbon ring of benzene or the condensed 6-carbon rings of other aromatic derivatives such as naphthalene) also referred to as derivatives of arenes, usually missing an H where they are bound to Si in POSS or to some other molecule, heteroaryl groups (either a 6-membered or 5-membered aromatic ring containing one or more atoms other than carbon in the ring, e.g. N, S or O, or structures containing condensed heteroaromatic rings) acyl groups (organic acids without the OH group, e.g., CH₃CO— or C₆H₅CO—), alkoxy groups (alkyl radicals attached to the remainder of a molecule by oxygen), such as methoxy, ester groups, acid groups, acrylate groups, alkyl acrylate groups, hydroxy groups, halogens, amino groups, alkylamino groups, aminoalkyl groups, groups containing one or more tertiary or quaternary nitrogens, silicone containing groups, sulfur containing groups, epoxides, azo groups, diazo groups, halogens, cyclic compounds which can undergo ring opening polymerization or ring opening metathesis polymerization. R groups may also be monomers or polymers where POSS will be used as a pendant substituent of the polymer. Acrylates and cationic polymers providing conditioning properties are provided in some embodiments.

Where appropriate, any of these R groups may themselves be substituted or unsubstituted, saturated or unsaturated, linear or branched. Possible substitutions include C₁-C₃₀ alkyl groups, C₁-C₃₀ alkenyl groups, C₁-C₃₀ alkynyl groups, C₆-C₁₈ aryl groups, acyl groups, alkoxy or other groups, carboxy groups, ester groups, acrylate groups, alkyl acrylate groups, trihydroxy groups, amino groups, alkylamino groups including mono and dialkylamino groups, mono and dihydroxy alkylamino groups, cyano groups, aminoalkyl groups, groups containing one or more tertiary or quaternary nitrogens, silicone containing groups, sulfur and/or phosphorous containing groups, SO₂X, SO₃X, where X is H, methyl or ethyl, epoxides and epoxide containing groups, azo groups, diazo groups, halogens, cyclic compounds which can undergo ring opening polymerization or ring opening metathesis polymerization. Indeed, any group which can be attached to a corner of a POSS molecule can be used.

When these R groups are carbon containing fatty acids or fatty alcohols, aromatic or cyclic groups, they generally may contain between 6 and 50 carbon atoms and may be saturated or unsaturated, substituted as discussed above or unsubstituted and branched or linear, as appropriate for a given group.

More specifically, possible R groups include, without limitation, hydroxy groups including mono or poly hydroxy groups, phenols, alkoxy, hydroxy alkyls, silanes, amino and in particular, quats, halosilanes, epoxides, alkyl carbonyls, alkanes, haloalkyls, halogens, acrylates, methacrylates, thiols, nitriles, norbornenyls, branched alkyl groups, polymers, silanes, silanols, styryls and thiols. In a single POSS molecule of Formula I, R₁ could be H, R₂—OH, R₃—NH₂, R₄—CH₂CH₂N⁺CH₃(OCH₂CH₃)CH₂CH₂CH₃, R₅—CH₂CH₂CHOCH₂ (epoxide), R₆—OC(CH₃)₃, R₇—OOC(CH₂)₁₆CH₃ and R₈ could be Cl. This is a hypothetical example, merely to illustrate that each of the R groups can be derivatized separately and to emphasize the wide variety of possible substitutions.

In some embodiments, these POSS molecules are not completely substituted with the same R groups (e.g., not all R₁-R₆, R₁-R₈, R₁-R₁₀ or R₁-R₁₂ (and any other R groups, as appropriate, given the number of Si atoms and available bonds in a given POSS molecule) are methyl, isobutyl or phenyl). This is particularly preferred for POSS molecules that have the structure of Formula I. Moreover, when a POSS molecule having 8 Si subunits, as depicted in Formula I, is employed, at least one of the R groups is a group other than a methyl, particularly where the silicon resin is a T resin and, even more particularly, Resin MK.

Also contemplated under the term POSS is the family of commercially available compounds available from Hybrid Plastics, 55 W.L. Runnels Industrial Drive Hattiesburg, Miss. 39401 and Mayaterials, Inc. P.O. Box 87, South Lyon, Mich. 48178-0087. According to the manufacturers, these commercially available materials break down into several general categories:

In some embodiments, the POSS used in the coatings (nail coating or nail topcoat) of the present embodiments has the formula of (C₆H₁₁O₂)ₙ(SiO_{1.5})ₙ, where n is 6 (see Formula III), 8 (see Formula I), 10 (see Formula IVA), or 12 (see Formula IVC) and C₆H₁₁O₂ represents a glycidyl epoxide having the structure:

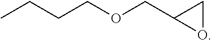

In some embodiments, the POSS used in the coatings of the present embodiments has the formula of (C₆H₁₁O₂)ₙ(SiO_{1.5})ₙ, where n is 8, 10, or 12. In some embodiments, the POSS used in the coatings of the present embodiments has formula of (C₆H₁₁O₂)ₙ(SiO_{1.5})ₙ, where n is 8 or 10. In some embodiments, the POSS used in the coatings of the present embodiments has the formula of (C₆H₁₁O₂)ₙ(SiO_{1.5})ₙ, where n is 8. In some embodiments, the POSS used in the coatings of the present embodiments is a mixture of POSS structures having the formula (C₆H₁₁O₂)ₙ(SiO_{1.5})ₙ, where n is 6, 8, 10, and 12. In some embodiments, the POSS used in the coatings of the present embodiments is a mixture of POSS structures having the formula (C₆H₁₁O₂)ₙ(SiO_{1.5})ₙ, where n is 8, 10 and 12. In some embodiments, the POSS used in the coatings of the present embodiments is a mixture of POSS structures having the formula of (C₆H₁₁O₂)ₙ(SiO_{1.5})ₙ, where n is 8 and 10.

In some embodiments, the POSS molecules are functionalized with at least one group or a plurality of groups. Examples of functional groups on the polymer and POSS materials include, but are not limited to, functional silicones—for example, hydroxy, urethane, acrylate, vinyl, Si—H, amides, MQ or T groups, functional acrylates, functional polyamides, PVK, PVA, PS, PEG, PPG, polysaccharides or modified starch, functional block copolymers, functional polyesters and polyethers, fluorinated polymers and wax to bring about the cross-linking reaction between the polymer chains and POSS materials to provide desired properties.

Examples of suitable POSS compounds include, but are not limited to, dodecaphenyl, octaisobutyl and octamethyl POSS. POSS hybrid chemical compounds have molecular-level functional ingredients and are commercially available from Hybrid Plastics (Fountain Valley, Calif.). More specifically, the POSS compounds include, but are not limited to, the following: 1-[3-(allylbisphenol A)propyldimethylsiloxy]-3,5,7,9,11,13,15heptacyclopentylpentacyclo-[9.5.1.13,9.15,15.17,13]octasiloxane; 1-[3-(allylbiphenol)propyldimethylsiloxy]3,5,7,9,11,13,15heptacyclopentylpentacyclo-[9.5.1.13,9.15,15.17,13]octasiloxane; 1-[3-(1,3-propanediol-2-ethyl-2-methyloxy)propyldimethylsiloxy]-3,5,7,9,1-1,13,15-heptacyclopentylpentacyclo-[9.5.1.13,9.15,15.17,13]octasiloxane; 1-[(2-methyl,2-hydroxy)butyldimethylsiloxy]-3,5,7,9,11,13,15-heptacyclopentylpentacyclo-[9.5.1.13,9.15,15.17,13]octasiloxane; 1-[3-(ethoxydimethylsilyl)propyl]3,5,7,9,11,13,15-heptacyclopentylpentacyclo[9.5.1.13,9.15,15,17,13]octasiloxane; 1-[2-(diethoxymethylsilyl)propyl]-5,7,9,11,13,15-heptacyclopentylpentacyclo[9.5.1.13,9.15,15,17,13]octasiloxane; 1-[3-(triethoxysilyl)propyl]3,5,7,9,11,13,15-heptacyclopentylpentacyclo[9-.5.1.13,9.15,15,17,13]octasiloxane; 1-[2-(ethoxydimethylsilyl)ethyl]3,5,7,9,11,13,15-heptacyclopentylpentacyclo[9.5.1.13,9.15,15,17,13]octasiloxane; 1-[2-(diethoxymethylsilyl)propyl]3,5,7,9,11,13,15-heptacyclopentylpentacyclo[9.5.1.13,9.15,15,17,13]octasiloxane; 1-[2-(triethoxysiyl)propyl]3,5,7,9,11,13,15-heptacyclopentylpentacyclo[9.5.1.13,9.15,15,17,13]octasiloxane; POSS-BisPhenol A-urethanes; POSS-DiMethylol-urethanes; 1-chloro-3,5,7,9,11,13,15-heptacyclopentylpentacyclo[9.5.1.13,9.15,15.17,-13]octasiloxane; 1-[2-(chlorodimethylsilyl)ethyl]3,5,7,9,11,13,15heptacyclopentylpentacyclo[9.5.1.13,9.15,15.17,13]octasiloxane; 1-[2-(dichloromethylsilyl)ethyl]-3,5,7,9,11,13,15heptacyclopentylpentacyclo[9.5.1.13,9.15,15.17,13]octasiloxane; 1-[2-(trichlorosilyl)ethyl]-3,5,7,9,11,13,15-heptacyclopentylpentacyclo[9-.5.1.13,9.15,15.17,13]octasiloxane; 1-[3-(chlorodimethylsilyl)propyl]-3,5,7,9,11,13,15-heptacyclopentylpentacyclo-[9.5.1.13,9.15,15.17,13]octasiloxane; 1-[3-(dichloromethylsilyl)propyl]-3,5,7,9,11,13,15-heptacyclopentylpentacyclo[9.5.1.13,9.15,15.17,13]octasiloxane; 1-[3-(trichlorosilyl)propyl]-3,5,7,9,11,13,15-heptacyclopentylpentacyclo[-9.5.1.13,9.15,15.17,13]octasiloxane; 1,3,5,7,9,11,13,15-[2-(chlorodimethylsilyl)ethyl]pentacyclo[9.5.1.13,9,15-,15.17,13]octasiloxane; 1,3,5,7,9,11,13,15-[2-(chlorodimethylsilyl)ethyl]pentacyclo[9.5.1.13,9,15-,15.17,13]octasiloxane; 1,3,5,7,9,11,13,15-[2-(dichlorodimethylsilyl)ethyl]pentacyclo[9.5.1.13,9.15,15.17,13]octasiloxane; 1-[(2-epoxy)propyl]-3,5,7,9,11,13,15-heptacyclopentylpentacyclo[9.5.1.13,-9.15,15.17,13]octasiloxane; 1-[2-(cyclohexyl-3-epoxy)ethyl]-3,5,7,9,11,13,15-heptacyclopentylpentacyclo[9.5.1.13,9.15,15.17,13]octasiloxane; POSS-diepoxide resins; 1,3,5,7,9-octavinyl-11,13,15-epoxyethylpentacyclo[9.5.1.1.3, 9.1.15,15.1.1-7,13]octasiloxane; endo-3,7,14-tris[1-(3-dimethylsiloxy)propyloxy-2,3-epoxypropyl]-1,3,5,7,9-,11,14,-heptacyclopentyltricyclo[7.3.3.1,5,11]heptasiloxane; 1-(methylpropionato)-3,5,7,9,11,13,15-heptacyclopentylpentacyclo[9.-5.1.1-.3,9.15,15.17,13]octasiloxane; 1 (ethylundecanoato)-3,5,7,9,11,13,15-heptacyclopentylpentacyclo[9.-5.1.1.3, 9.15,15.17,13]octasiloxane; 1-[(3-chloro)propyl]-3,5,7,9,11,13,15-heptacyclopentylpentacyclo[9.5.1.13,9.15,15.17,13]octasiloxane; 1-[4-chlorophenyl]-3,5,7,9,11,13,15-heptacyclopentylpentacyclo[9.5.-1.13,-9.15,15.17,13]octasiloxane; 1-[chlorobenzyl]-3,5,7,9,11,13,15-heptacyclopentylpentacyclo[9.5.1.13,9.1-5,15.17,13]octasiloxane; 1-[2-(chlorobenzyl)ethyl]-3,5,7,9,11,13,15-heptacyclopentylpentacyclo[9.5.1.13,9.15,15.17,13]octasiloxane; 1-[3-(methacryl)propyl]-3,5,7,9,11,13,15-heptacyclopentacyclo[9.5.1.13,9.-15,15.17,13]octasiloxane; 1-[3-(methacryl)propyldimethylsiloxy]-3,5,7,9,11,13,15-heptacyclopentylpentacyclo-[9.5.1.13,9.15,15.17,13]-octasiloxane; 1-(3,3,3-trifluoropropyldimethylsiloxy)-1,3,5,9,11,13,15-heptacyclopentyl-7-[3-(methacryl)propyl]-7-methyltetracyclo[9.5.1.15,11.19,15]octasiloxane; 1-(tridecafluoro-1,1,2,2-tetrahydrooctyldimethylsiloxy)-1,3,5,9,11,13,15-heptacyclopentyl-7-[3-(methacryl)propyl]-7-methyltetracyclo[9.5.1.15,11,19-,15]octasiloxane; 1-(trimethylsiloxy)-1,3,5,9,11,13,15-heptacyclopentyl-7-[3-(methacryl)propyl]-7-ethyltetracyclo[9.5.1.15,11.19,15]octasiloxane; 1,3,5,7,9-pentavinyl-11,13,15-[1-hydroxy-2-(methacryl)ethyl]pentacyclo[9.-5.1.13,9.15,15.17,13]octasiloxane; 1,3,5,7,9,11-hexacyclohexyltetracyclo[5.5.1.13,11.15,9]hexasiloxane; 1,3,5,7,9,11,13,15-octacyclohexylpentacyclo[9.5.1.13,9.15,15.17,13]octasiloxane; 1,3,5,7,9,11,13,15-octacyclopentylpentacyclo[9.5.1.13,9.15,15,17,1-3]octasiloxane; 1,3,5,7,9,11,13,15-octaphenylpentacyclo[9.5.1.13,9.15,15.17,13]octasiloxane; 1,3,5,7,9,11,13,15-octamethylpentacyclo[9.5.1.13,9.15,15.17,13]octasiloxane; 1,3,5,7,9,11,13,15-octakis(dimethylsilyloxy)pentacyclo[9.5.1.13,9,1-.5,15.17,13]octasiloxane; POSS-modified Nylon 6; 1-[(3-cyano)propyl]-3,5,7,9,11,13,15-heptacyclopentylpentacyclo[9.5-.1.1.sup.3,9.15,15.17,13]octasiloxane; 1-[2-(Norbornen-2-yl)ethyl]-3,5,7,9,11,13,15-heptacyclopentylpentacyclo[9-.5.1.13,9.15,15.17,13]octasiloxane; 1-[2-(Norbornen-2-yl) ethyldimethylsiloxy]-3,5,7,9,11,13,15heptacyclopentylpentacyclo[9.5.1.13,-9.15,15.17,13]-octasiloxane; poly(ethylnorbornenylPOSS-co-norbornene); 1,1,3,3-(norbornenyldimethylsiloxy)-1,3,-dicyclohexyldisiloxane; 1-[3-(allylbisphenol A)propyldimethylsiloxy]-3,5,7,9,11,13,15 heptacyclopentylpentacyclo-[9.5.1.13,9.15,15.17,13]octasiloxane; 1-[3-(allylbiphenol)propyldimethylsiloxy]-3,5,7,9,11,13,15 heptacyclopentylpentacyclo-[9.5.1.13,9.15,15.17,13]octasiloxane; 1,3,5,7,9,11,13,15-octavinylpentacyclo[9.5.1.13,9.15,15.17,13]octasiloxane; 1-vinyl-3,5,7,9,11,13,15-heptacyclopentylpentacyclo[9.5.1.13,9.15,15,1-7,13]octasiloxane; 1-allyl-3,5,7,9,11,13,15-heptacyclopentylpentacyclo[9.5.1.13,9.15,15.17,13]octasiloxane; 1-[2-(cyclohexen-3-yl)ethyl]-3,5,7,9,11,13,15-heptacyclopentylpentacyclo[9.5.1.13,9.15,15.17,13]octasiloxane; poly(dimethyl-co-methylvinyl-co-methylethylsiloxyPOSS)siloxane; POSS-diepoxide resins; POSS-Bis Phenol A-urethanes; 1-[2(diphenylphosphino)ethyl]3,5,7,9,11,13,15-heptacyclopentylpentacyclo[-9.5.1.13,9.15,15.17,13]octasiloxane; 1-[2(diphenylphosphino)propyl]3,5,7,9,11,13,15-heptacyclopentylpentacyclo[9.5.1.13,9.15,15.17,13]octasiloxane; 1-hydrido-3,5,7,9,11,13,15-heptacyclopentylpentacyclo[9.5.1.13,9.15,15,17-,13]octasiloxane; 1-[hydridodimethylsiloxy]-3,5,7,9,11,13,15-heptacyclopentylpentacyclo[9.5-.1.13,9.15,15.17,13]octasiloxane; endo-3,7,14-tri(dimethylsilylhydrido)-1,3,5,7,9,11,14-heptacycopentyltricyclo[7.3.3.15,15,11]heptasiloxane; 1,1,3,3-(hydridodimethylsiloxy)-1,3-dicyclohexyldisiloxane; poly(dimethyl-co-methylhydrido-co-methylpropyl POSS)siloxanendo-3,7,14-trihydroxy-1,3,5,7,9,11,14-heptacyclopentyltricyclo[7.3.3.15,11]heptasiloxane; endo-3,7,14-trihydroxy-1,3,5,7,9,11,14-heptacyclohexyltricyclo[7.3.-3.15,-11]heptasiloxane; 1-hydroxy-3,5,7,9,11,13,15-heptacyclopentylpentacyclo[9.5.1.13,9.15,15,17-,13]octasiloxane; 1,1,3,3-(tetrahydroxy)-1,3-dicyclohexyldisiloxane; 1,3,5,7-(tetrahydroxy)-1,3,5,7-(tetraphenyl)cyclotetrasiloxane; endo-7,14-dihydroxy-3-(3,3,3-trifluoropropyldimethylsiloxy)-1,3,5,9,11,13,15-heptacyclopentyltricyclo[7.3.3.15,11]octasiloxane; 1,3,5,7-(tetrahydroxy)-1,3,5,7,-(tetraphenyl)cyclotetrasiloxane; endo-7,14,-dihydroxy-3-(3,3,3-trifluoropropyldimethylsiloxy)-1,3,5,9,11,13,15-eptacyclopentyltricyclo[7.3.3.1.sup.5,11]octasiloxane; 1-[2-(styryl)ethyldimethylsiloxy]-3,5,7,9,11,13,15-heptacyclopentylpentacyclo[9.5.1.13,9.15,15.17,13]-octasiloxane; 1-[(4-vinyl)phenyl]-3,5,7,9,11,13,15-heptacyclopentylpentacyclo[9.5.1.13,-9.15,15.17,13]octasiloxane; 1-[2-(styryl)ethyl]-3,5,7,9,11,13,15-heptacyclopentylpentacyclo[9.5.1.13,-9.15,15.17,13]-octasiloxane; R=cyclopentyl, TMP DiolCyclopentyl-POSS; R=i-butyl, Trans-CyclohexaneDiolisobutyl-POSS; R=i-butyl, 1,2-PropaneDiolisobutyl-POSS; R=i-butyl, Aminopropylisobutyl-POSS; R=i-octyl, Aminopropylisobutyl-POSS; R=i-butyl, Aminoethylaminopropylisobutyl-POSS; R=Cyclopentyl, IsocyanatopropyldimethylsilylCyclopentyl-POSS; R=i-butyl, MethacrylisobutylTitanium-POSS; OctaAmmonium-POSS; OctaAmmonium-POSS; Poly(styryl POSS-co-styrene); poly(vinylsilsesquioxane); and structures having 10 and 12 silicon atoms in the cage.

The POSS of the present invention may be prepared by hydrolytic condensation reactions of trifunctional organosilicone monomers, e.g. RSi(OMe). Methods of preparing POSS are described in U.S. Pat. No. 8,133,478 and U.S. Pat. No. 6,372,843, which are incorporated herein by reference in their entireties.

In some embodiments, the POSS used in the coatings of the present embodiments is EP0409 POSS (Hybrid Plastics), which is a blend of caged and non-caged structures as described in, for example, U.S. Pat. Nos. 6,716,919 and 6,927,270, each of which is incorporated herein by reference in its entirety.

### POSS Molecular Silicas™

POSS Molecular Silicas™ possess a robust Si—O core surrounded by non-reactive organic groups (R₁-R₈) which permit the inorganic core to be compatible with an organic matrix. This allows POSS Molecular Silicas™ to be compounded into standard polymers yielding true nanocomposites with complete molecular level dispersion. The unique ability of POSS Molecular Silicas™ to be dispersed at the molecular level is the key to reinforcing polymer segments and coils leading to significant property enhancements.

### POSS Silanols

POSS Silanols possess a hybrid inorganic-organic three-dimensional structure which contains from one to four stable silanol (Si—OH) groups.

### POSS Functionalized Monomers

POSS Functionalized Monomers possess a hybrid inorganic-organic three-dimensional structure which contains from one to eight reactive organic functional groups. The majority of POSS Functionalized Monomers contain seven non-reactive organic groups with one unique functionality. The unique functional groups that are currently available include, but are not limited to, amines, esters, epoxides, methacrylates, olefins, silanes, styryls, and thiols. By varying the functional group and the seven non-reactive organic groups, a multitude of POSS Functionalized Monomers can be prepared to meet almost any need. While the monofunctional POSS Monomers can be incorporated by copolymerization or grafting, multifunctional POSS Monomers can be utilized as effective cross-linkers. POSS Functionalized Monomers react similarly in polymerization, grafting and cross-linking reactions to standard organic monomers. While they react like simple organic monomers, when incorporated into a polymeric material, POSS Functionalized Monomers impart significant improvements in the thermal, mechanical, and gas separation properties of traditional plastics.

### POSS Polymers and Resins

POSS Polymers and Resins possess a hybrid inorganic-organic composition and can be either thermoplastic or thermoset materials. As a class of materials, POSS Polymers and Resins are comprised of either (1) polymers in which a POSS Functionalized Monomer has been co-polymerized or grafted onto a polymer chain, or (2) silsesquioxane resins possessing some cage structure (See, e.g. Formula X). POSS Polymers and Resins can be used as stand-alone replacements for traditional materials or they may be compounded or solution blended into traditional polymeric materials to enhance the properties of the base resin. The types of POSS Polymers and Resins that are currently available include, but are not limited to, silicones, styrenics, acrylics, and norbornenes.

POSS molecules available from Hybrid Plastics include, without limitation, those based on Formulas I-IV, and are selected from alcohols and phenols (such as TMP DiolCyclopentyl-POSS, TMP DiolIsobutyl-POSS, Trans-CyclohexaneDiolCycohexyl-POSS, Trans-CyclohexaneDiolIsobutyl-POSS, 1,2-PropaneDiolCyclohexyl-POSS, 1,2-PropaneDiolIsobutyl-POSS, and OctaHydroxypropyldimethylsilyl-POSS), alkoxysilanes (such as DiethoxymethylsilylethylCycohexyl-POSS, DiethoxymethylsilylethylIsobutyl-POSS, DiethoxymethylsilylpropylCyclohexyl-POSS, DiethoxymethylsilylpropylIsobutyl-POSS, EthoxydimethylsilylethylCyclohexyl-POSS, EthoxydimethylsilylethylIsobutyl-POSS, EthoxydimethylsilylpropylCyclohexyl-POSS (may contain some isomer), EthoxydimethylsilylpropylIsobutyl-POSS (may contain some β-isomer), TriethoxysilylethylCyclohexyl-POSS, TriethoxysilylethylIsobutyl-POSS, TriethoxysilylpropylCyclohexyl-POSS (may contain some β-isomer) and TriethyoxysilylpropylIsobutyl-POSS (may contain some β-isomer)), amines (such as AminopropylCyclohexyl-POSS, AminopropylIsobutyl-POSS, AminopropylIsooctyl-POSS, AminoethylaminopropylCyclohexyl-POSS, AminoethylaminopropylIsobutyl-POSS, OctaAminophenyl-POSS and OctaAmmonium-POSS), chlorosilanes (such as MonoChloroCyclohexyl-POSS, MonoChloroCyclopentyl-POSS, MonoChloroIsobutyl-POSS, ChlorodimethylsilylethylCyclohexyl-POSS, ChlorodimethylsilylethylIsobutyl-POSS, ChlorodimethylsilylpropylCyclohexyl-POSS (may contain some β-isomer), chlorodimethylsilylpropylIsobutyl-POSS (may contain some β-isomer), DichloromethylsilylethylCyclohexyl-POSS, DichloromethylsilylethylIsobutyl-POSS, DichloromethylsilylpropylCyclohexyl-POSS (may contain some β-isomer), DichloromethylsilylpropylIsobutyl-POSS (may contain some β-isomer), TrichlorosilylethylCyclohexyl-POSS, TrichlorosilylethylIsobutyl-POSS, TrichlorosilylpropylCyclohexyl-POSS (may contain some β-isomer), TrichlorosilylpropylIsobutyl-POSS (may contain some β-isomer), Octa(chlorosilylethyl)-POSS (may contain some β-isomer), Octa(dichlorosilylethyl)-POSS (may contain some β-isomer) and Octa(trichlorosilylethyl)-POSS (may contain some β-isomer)), epoxides (such as EpoxyCyclohexylCyclohexyl-POSS, EpoxyCyclohexylCyclopentyl-POSS, EpoxyCyclohexylIsobutyl-POSS, EpoxyCyclohexylDisilanolIsobutyl-POSS, EpoxyCyclohexyl-POSS Cage Mixtures such as EpoxypropylCyclopentyl-POSS, EpoxypropylIsobutyl-POSS, GlycidylCyclohexyl-POSS, GlycidylCyclopentyl-POSS, GlycidylEthyl-POSS, GlycidylIsobutyl-POSS, GlycidylIsoctyl-POSS, GlycidylPhenyl-POSS, OctaEpoxyCyclohexyldimethylsilyl-POSS, OctaGlycidyldimethylsilyl-POSS, TrisGlycidylCyclohexyl-POSS, TrisGlycidylCyclopentyl-POSS, TrisGlycidylEthyl-POSS and TrisGlycidylIsobutyl-POSS), esters (such as EthylUndecanoateCyclohexyl-POSS, EthylUndecanoateCyclopentyl-POSS, EthylUndecanoateIsobutyl-POSS, MethylPropionateCyclohexyl-POSS, MethylPropionateCyclopentyl-POSS and MethylPropionateIsobutyl-POSS), fluoroalkyls (such as Fluoro(3)DisilanolCyclopentyl-POSS, Fluoro(13)DisilanolCyclopentyl-POSS, Fluoro(13)DisilanolIsobutyl-POSS, MethacrylFluoro(3)Cyclopentyl-POSS mixture of isomers, MethacrylFluoro(13)Cyclopentyl-POSS mixture of isomers, MethacrylFluoro(3)Isobutyl-POSS mixture of isomers, DodecaTrifluoropropyl-POSS, TriFluoroCyclohexyl-POSS, TriFluoroCyclopentyl-POSS, TriFluoroIsobutyl-POSS, TrifluoropropylIsobutyl-POSS, TrisFluoro(3)Cyclopentyl-POSS and TrisFluoro(13)Cyvlopentyl-POSS), halides (such as ChlorobenzylCyclohexyl-POSS, ChlorobenzylCyclopentyl-POSS, ChlorobenzylIsobutyl-POSS, ChlorobenzylethylCyclohexyl-POSS, ChlorobenzylethylCyclopentyl-POSS, ChlorobenzylethylIsobutyl-POSS, ChlorophenylCyclohexyl-POSS, ChlorophenylCyclopentyl-POSS, ChlorophenylIsobutyl-POSS, ChlorophenylPhenyl-POSS, ChloropropylCyclohexyl-POSS, ChloropropylCyclopentyl-POSS and ChloropropylIsobutyl-POSS), isocyanates (such as IsocyanatopropyldimethylsilylCyclohexyl-POSS and IsocyanatopropyldimethylsilylIsobutyl-POSS), methacrylates and acrylates (such as AcryloCyclohexyl-POSS, AcryloCyclopentyl-POSS, AcryloIsobutyl-POSS, MethacrylCyclohexyl-POSS, MethacrylCyclopentyl-POSS, MethacrylEthyl-POSS, MethacrylIsobutyl-POSS, MethacrylIsooctyl-POSS 90%, MethacrylPhenyl-POSS, MethacrylDisilanolCyclohexyl-POSS, MethacrylDisilanolCyclopentyl-POSS, MethacrylDisilanolIsobutyl-POSS, MethacrylFluoro(3)Cyclopentyl-POSS, MethacrylFluoro(13)Cyclopentyl-POSS, MethacryltrimethylsiloxyCyclopentyl-POSS, MethacryltrimethylsiloxyIsobutyl-POSS, Methacryl-POSS Cage Mixture, OctaMethacryldimethylsilyl-POSS, TrisMethacrylCyclohexyl-POSS and TrisMethacrylIsobutyl-POSS), molecular silica (such as DodecaPhenyl-POSS, DodecaPhenyl-POSS, 85%, Isooctyl-POSS Cage Mixture 95%, OctaCyclohexyl-POSS, OctaCyclopentyl-POSS, OctaIsobutyl-POSS, OctaMethyl-POSS, OctaPhenyl-POSS, OctaTMA-POSS, DodecaTrifluoropropyl-POSS, OctaTrimethylsiloxy-POSS, Phenethyl-POSS Cage Mixture and PhenethylIsobutyl-POSS), nitriles (such as CyanoethylCyclohexyl-POSS, CyanoethylCyclopentyl-POSS, CyanoethylIsobutyl-POSS, CyanopropylCyclohexyl-POSS, CyanopropylCyclopentyl-POSS and CyanopropylIsobutyl-POSS), norbornenyls (such as NorbornenylethylCyclohexyl-POSS, NorbornenylethylIsobutyl-POSS, NorbornenylethylDiSilanolCyclohexyl-POSS, NorbornenylethylDiSilanolCyclopentyl-POSS, NorbornenylethylDiSilanolIsobutyl-POSS, Tris NorbornenylCyclohexyl-POSS, Tris NorbornenylCyclopentyl-POSS and Tris NorbornenylIsobutyl-POSS), olefins (such as AllylCyclohexyl-POSS, AllylCyclopentyl-POSS, AllyIsobutyl-POSS, AllylDimethylsilylCyclopentyl-POSS, CyclohexenylethylCyclopentyl-POSS, DimethylvinylCyclopentyl-POSS, DiphenylvinylCyclopentyl-POSS, MonoVinylCyclohexyl-POSS, MonoVinylCyclopentyl-POSS, MonoVinylIsobutyl-POSS, PhenylMethylVinylCyclopentyl-POSS, Tris(Dimethylvinyl)Isobutyl-POSS, TrivinylsilylCyclopentyl-POSS, OctaCyclohexenyldimethylsilyl-POSS, OctaVinyldimethylsilyl-POSS, OctaVinyl-POSS and Vinyl-POSS Cage Mixture), phosphines (such as DiphenylphosphinoethylCyclopentyl-POSS and DiphenylphosphinopropylCyclopentyl-POSS), polymers (such as Poly(dimethyl-co-methylhydrido-co-methylpropylPOSS)siloxane, Poly(dimethyl-co-methylvinyl-co-methylethylsiloxyPOSS)siloxane, OctaMethyl-POSS Nanoreinforced™ Polypropylene, 10 wt %, Poly(ethylsilsesquixane) uncured, Poly(methylsilsesquioxane) uncured, Poly(phenylsilsesquioxane) uncured, Poly(propylmethacrylPOSS-co-methylmethacrylate), Poly(propylmethacrylPOSS-co-styrene), Poly(styrylPOSS-co-styrene), Poly(vinylsilsesquioxane) uncured and Poly(vinylsilsesquioxane) fully cured FireQuench™), silanes (such as DimethylsilaneCyclohexyl-POSS, DimethylsilaneCyclopentyl-POSS Schwab Hydride, DimethylsilaneIsobutyl-POSS, MonoSilaneCyclohexyl-POSS, MonoSilaneIsobutyl-POSS, OctaSilane-POSS, Tris(Dimethylsilane)Cyclohexyl-POSS, Tris(Dimethylsilane)Cyclopentyl-POSS and Tris(Dimethylsilane)CycloIsobutyl-POSS), silanols (such as CyclohexenyldimethylsilylDisilanolIsobutyl-POSS, DimethylphenylDisilanolCyclopentyl-POSS, DimethylvinylDisilanolCyclohexyl-POSS, DimethylvinylDisilanolCyclopentyl-POSS, DimethylvinylDisilanolIsobutyl-POSS, DiSilanolCyclopentyl-POSS, DiSilanolIsobutyl-POSS, EpoxyCyclohexylDisilanolIsobutyl-POSS, Fluoro(3)DisilanolCyclopentyl-POSS, Fluoro(13)DisilanolCyclopentyl-POSS, Fluoro(13)DisilanolIsobutyl-POSS, MethacrylDisilanolCyclohexyl-POSS, MethacrylDisilanolCyclopentyl-POSS, MethacrylDisilanolIsobutyl-POSS, MonoSilanolCyclohexyl-POSS, MonoSilanolCyclopentyl-POSS Schwabinol, MonoSilanolIsobutyl, NorbornenylethylDiSilanolCyclohexyl-POSS, NorbornenylethylDiSilanolCyclopentyl-POSS, NorbornenylethylDiSilanolIsobutyl-POSS, TMS DiSilanolCyclohexyl-POSS, TMS DiSilanolIsobutyl-POSS, TriSilanolCyclohexyl-POSS, TriSilanolCyclopentyl-POSS, TirSilanolEthyl-POSS, TriSilanolIsobutyl-POSS, TriSilanolIsooctyl-POSS and TriSilanolPhenyl-POSS), styrenes (such as StyrenylIsobutyl-POSS, StyrylCyclohexyl-POSS, StyrylCyclopentyl-POSS and StyrylIsobutyl-POSS), and thiols (such as MercaptopropylCyclohexyl-POSS, MercaptopropyIsobutyl-POSS and MercaptopropylIsooctyl-POSS 90%). Other POSS products may be purchased from ALDRICH. Still others are described in U.S. Pat. No. 8,133,478 and U.S. Pat. No. 5,047,492, the text of which, and in particular, the POSS molecules described in the passage of column 1, line 22 through column 2, line 48, are hereby incorporated by reference and U.S. Pat. No. 2,465,188, the text of which is also hereby incorporated by reference. See also U.S. Pat. No. 5,858,544, the text of which is also incorporated by reference.

Particularly preferred POSS molecules useful for producing coating compositions in accordance with the present embodiments include: TrisFluoro(13)Cyclopentyl-POSS (Cat. No. FL0590; C₆₅H₉₃F₃₉O₁₂Si₁₀; Mw: 2088.24 g/mole); MercaptopropylIsobutyl-POSS (Cat. No. TH1550; C₃₁H₇₀O₁₂Si_{g}; Mw: 891.63 g/mole); MercaptopropylIsooctyl-POSS (Cat. No. TH1555; C₅₉H₁₂₆O₁₂Si₈; Mw: 1284.37 g/mole); Poly(methacrylpropylisooctylPOSS-co-methymethacrylate) 60% wt (Cat. No. PM1275.4-60; (R₇O₁₄Si₈)60-co-(C₅H₈O₂)₄₀); Poly(MethacrylpropylisooctylPOSS-co-methylmethacrylate) 80% wt. (Cat. No. PM1275.4-80; (R₇O₁₄Si₈)₈₀-co-(C₅H₈O₂)₂₀); OctaIsobutyl-POSS (Cat. No. MS0825; C₃₂H₇₂O₁₂Si₈; Mw: 873.60 g/mole); OctaPhenyl-POSS (Cat. No. MS0840; C₄₈H₄₀O₁₂Si₈; Mw: 1033.53 g/mole); Isooctyl-POSS Cage Mixture, 95% (Cat. No. MS0805; [Me₃CCH₂CH(Me)CH₂]ₙTₙ n=8; C₆₄H₁₃₆O₁₂Si₈; Mw: 1322.46 g/mole based on n=8); EpoxyCyclohexylCyclohexyl-POSS (Cat. No. EP0399; C₅₀H₉₀O₁₃Si₈; Mw: 1123.93 g/mole); EpoxyCyclohexylIsobutyl-POSS (Cat. No. EP0402; C₃₆H₇₆O₁₃Si₈; Mw: 941.66 g/mole); Glycidyl POSS Cage Mixture (Cat. No. EP0409); GlycidylCyclohexyl-POSS (Cat. No. EP0415; C₄₈H₈₈O₁₄Si₈; Mw: 1113.89 g/mole); GlycidylIsobutyl-POSS (Cat. No. EP0418); C₃₄H₇₄O₁₄Si₈; Mw: 931.63 g/mole); TrisGlycidylCyclohexyl-POSS (Cat. No. EP0421; C₆₆H₁₂₈O₁₈Si₁₀; Mw: 1490.57 g/mole); and OctaEpoxyCyclohexyldimethylsilyl-POSS (Cat. No. EP0430; C₈₀H₁₅₂O₂₈Si₁₆; Mw: 2011.41 g/mole); OctaAminophenyl-POSS (Cat. No. AM0280; C₄₈H₄₈N₈O₁₂Si₈; Mw: 1153.63 g/mole); OctaAminophenyl-POSS (Cat. No. AM0285; C₂₄H₇₂CL₈N₈O₁₂Si₈; Mw: 1173.18 g/mole); and OctaTMA-POSS (Cat. No. MS0860; C₃₂H₉₆O₂₀Si₈˜60H₂O; Mw: 2218.75 g/mole). These POSS molecules can be purchased from Hybrid Plastics, 55 W.L. Runnels Industrial Drive Hattiesburg, Miss. 39401, USA and Mayaterials.

As previously noted and as reflected in the patents and publications previously incorporated by reference, there are many known POSS molecules and many known ways to produce POSS compounds and various derivatives and polymers thereof. In general, however, one process of producing POSS includes the following steps: a) providing a trifunctional polyhedral oligomeric silsesquioxane of the formula Si₇R₇O₉(OA)₃, where OA is —OH, —OS_{b}(CH₃)₄, —OSn(CH₃)₃, or —OTI, and R is an alkyl, alkenyl, aryl, alkoxy group or other R group described herein; and b) corner capping said trifunctional polyhedral silsesquioxane by reacting said trifunctional polyhedral silsequioxane with a compound of the formula M-Z to form a polyhedral oligomeric silsesquioxane having the formula Si₇R₇O₁₂M_{(z)}. M is a silane, siloxane or organometallic group and Z is a reactive group selected from the group consisting of chloride, bromide or iodide. The process further includes the step of adding silver perchlorate to a solution of the polyhedral oligomeric silsesquioxane in aqueous acetone to convert reactive group Z to an alcohol. See U.S. Pat. No. 5,484,867. POSS molecules may also be made as described in a paper entitled “Polyhedral Oligosilsesquioxanes and Heterosilsesquioxanes” by Frank J. Feher of the Department of Chemistry of the University of California at Irvine, Calif. 92697-2025, USA, available from Gelest, Inc., the test of which is hereby incorporated by reference.

Mixtures of POSS molecules are specifically contemplated. Indeed, mixtures of POSS molecules with EPOSS molecules containing nine or more Si atoms within their cage-like structure are also contemplated. EPOSS molecules are also available commercially from Hybrid Plastics.

### Plasticizers

The nail coating of the present embodiments may also include an amount of plasticizer, which can be chosen by a person skilled in the art on the basis of his or her general knowledge, so as to obtain a composition which has cosmetically acceptable properties.

Plasticizers useful in the presently disclosed nail coating composition include plasticizers commonly employed in nail enamel compositions. These plasticizers encompass, but are not limited to, dibutyl phthalate, dioctyl phthalate, tricresyl phthalate, butyl phthalate, dibutoxy ethyl phthalate, diamylphthalate, tosyl amide, N-ethyl-tosyl amide, sucrose acetate isobutyrate, camphor, castor oil, citrate esters, glyceryl diesters, glyceryl triesters, tributyl phosphate, tri-phenyl phosphate, butyl glycolate, benzyl benzoate, butyl acetyl ricinoleate, butyl stearate, and dibutyl tartrate.

Additional examples of plasticizers suitable for use in the present invention, alone or as a mixture, include: glycols and derivatives thereof such as diethylene glycol ethyl ether, diethylene glycol methyl ether, diethylene glycol butyl ether, diethylene glycol hexyl ether, ethylene glycol ethyl ether, ethylene glycol butyl ether and ethylene glycol hexyl ether; glycerol esters; propylene glycol derivatives including propylene glycol phenyl ether, propylene glycol diacetate, dipropylene glycol butyl ether, tripropylene glycol butyl ether, propylene glycol methyl ether, dipropylene glycol ethyl ether, tripropylene glycol methyl ether, diethylene glycol methyl ether and propylene glycol butyl ether; acid esters, including carboxylic acid esters, such as citrates, phthalates, adipates, carbonates, tartrates, phosphates and sebacates; and oxyethylenated derivatives, including oxyethylenated oils, for example, plant oils such as castor oil; and mixtures thereof.

According to some embodiments, the plasticizer is a biodegradable plasticizer. In some embodiments, the plasticizer is an acetylated monoglyceride.

In some embodiments, the plasticizer is an alkyl citrate. In some embodiments, the alkyl citrates contemplated for use in the present embodiments are those resulting from esterification of citric acid with alcohols containing three or more carbon atoms, for example, tripropyl citrate, tributyl citrate, trihexyl citrate, etc. These esters may be derived from either primary or secondary alcohols. Esters derived from glycol and glycerol ethers containing one or more unetherified hydroxyl groups are also suitable plasticizers of similar characteristics.

In some embodiments, a plasticizer used in the present invention may be the mixture of acetyl tributyl and N-ethyl tosyl amide. The plasticizer may, for example, be present in an amount of from about 3% to about 12% by weight relative to the weight of the composition.

In some embodiments, the plasticizer is triethyl citrate (TEC). In some embodiments, the plasticizer is acetyl triethyl citrate (ATEC). In some embodiments, the plasticizer is tributyl citrate (TBC). In some embodiments, the plasticizer is acetyl tributyl citrate (ATBC). In some embodiments, the plasticizer is trioctyl citrate (TOC). In some embodiments, the plasticizer is acetyl trioctyl citrate (ATOC). In some embodiments, the plasticizer is trihexyl citrate (THC). In some embodiments, the plasticizer is acetyl trihexyl citrate (ATHC). In some embodiments, the plasticizer is butyryl trihexyl citrate (BTHC, trihexyl o-butyryl citrate). In some embodiments, the plasticizer is trimethyl citrate (TMC). In some embodiments, the plasticizer is alkyl sulphonic acid phenyl ester (ASE). In some embodiments, the plasticizer is vinyl chloride copolymer. In some embodiments, the plasticizer is 1,2-cyclohexane dicarboxylic acid diisononyl ester.

In some embodiments, the plasticizer is a tri-lower alkyl citrate. This includes triethyl citrate, tributyl citrate and triamyl citrate.

In some embodiments, the plasticizer is an acyl tri(lower alkyl) citrate where the alkyl group contains 2-4 carbon atoms. This includes acetyl triethyl citrate and acetyl tributyl citrate.

### Color Agents

“Colorants” or “coloring agents” useful in the cosmetic compositions of the invention may include, for example, pigments, including nacreous pigments, solid particles (for example, glitter flakes) and liposoluble colorants. Pigments may be white, transparent or colored, and mineral and/or organic. Among the mineral pigments which may be mentioned are titanium dioxide, optionally surface-treated, zirconium oxide or cerium oxide, and iron oxide or chromium oxide, manganese violet, ultramarine blue, chromium hydrate and ferric blue. Among the organic pigments which may be mentioned are carbon black, pigments of D&C type, and lakes based on cochineal carmine or on barium, strontium, calcium or aluminum.

Conventional coloring agents can be used, and examples include inorganic pigments such as titanium dioxide, iron oxides, titanated mica, iron oxide coated mica, ultramarine, chromium oxide, chromium hydroxide, manganese violet, bismuth oxychloride, guanine, and aluminum; pearlescent materials; and organic coloring agents such as ferric ammonium ferrocyanide, and D&C Red Nos. 6, 7, 34; Blue No. 1; Violet No. 2; and Yellow No. 5.

In some embodiments, the color agent is one or more lake pigments. A “lake pigment” is a pigment manufactured by precipitating a dye with an inert binder, or “mordant”, usually a metallic salt. The metallic salt or binder used is typically inert and insoluble in the vehicle, and is typically white or very neutral. In some embodiments, the metallic salt or binder has low tinting strength so that the dye itself determines which wavelengths are absorbed and reflected by the resulting precipitate.

The colorant can also include one or more pigments. These pigments can be white or colored, and inorganic or organic. Examples of inorganic pigments include titanium dioxide, which has optionally been surface-treated, zirconium oxide and cerium oxide, as well as iron oxide and chromium oxide, manganese violet, ultramarine blue, chromium hydrate and ferric blue, and metallic pigments such as aluminum and bronze. Examples of organic pigments include carbon black, pigments of D&C type and lakes based on cochineal carmine, barium, strontium, calcium, aluminum, and guanine.

The nacreous pigments can be chosen from white nacreous pigments such as mica coated with titanium or with bismuth oxychloride, colored nacreous pigments such as titanium mica with, for example, iron oxides, ferric blue, chromium oxide, or with an organic pigment of the above-mentioned type, as well as nacreous pigments based on bismuth oxychloride.

The inorganic pigments may be surface-treated as is customary to prevent migration or striation. Silicones and polyethylenes are most often used as the coatings for inorganic pigments and thus may be used according to the present invention. Colorant materials may also include chips or powder of mica or diamonds in the nail composition. Also useful are specialty materials giving rise to two-tone color effects such as liquid crystal silicones or multi-lamellar metallic particulates, which generally can be mixed with pigments or dyes to obtain a broader spectrum of brilliant color and increased luminous reflectance. Such materials are described in, e.g., U.S. Pat. Nos. 3,438,796; 4,410,570; 4,434,010; 4,838,648; 4,930,866; 5,171,363; 5,364,467; 5,569,535; 5,607,904; 5,624,486; 5,658,976; 5,688,494; 5,766,335; N. Hatberle et al., “Right and Left Circular Polarizing Colorfilters made from Crosslinkable Cholesteric LC-Silicones,” Conference Record of the 1991 International Display Research Conference (IEEE), pp. 57-59; R. Maurer et al., “Polarizing Color Filters made from Cholesteric LC-Silicones,” SID 90 Digest (1990), pp. 110-113; H.-J. Eberle et al., “Inverse Angle Dependence of the Reflection Colours of Cholesteric Polymeric Liquid Crystals Mixed with Pigments,” Liquid Crystals, 5(3), (1989), pp. 907-916; J. Pinsl et al., “Liquid Crystalline Polysiloxanes for Optical Once-Write Storage,” J. Molec. Electr., Vol. 3 (1987), pp. 9-13; and D. Makow, “Reflection and Transmission of Polymer Liquid-Crystal Coatings and their Application to Decorative Arts and Stained Glass,” Color Res. Applic. Vol. 11, No. 3, (1986), pp. 205-208, all of which are incorporated herein by reference in their entirety.

### Solvents

In some embodiments, the solvent used in the coatings of the present embodiments is an organic solvent. In some embodiments, the solvent used in the coatings of the present embodiments is a polar organic solvent. Non-limiting solvents include acetone, butyl acetate, isopropyl alcohol, ethanol, ethyl acetate, methyl ethyl ketone, and mixtures thereof. In some embodiments, the solvent is acetone. In some embodiments, the solvent is butyl acetate. In some embodiments, the solvent is ethyl acetate. In some embodiments, the solvent is butyl acetate, ethyl acetate, and mixtures thereof.

In some embodiments, the solvent is acetone, butyl acetate, butylene glycol, dipropylene glycol, disiloxane, ethyl acetate, ethyl ether, heptane, hexylene glycol, ethanol (denatured) isopropyl alcohol, limonene, n-butyl alcohol, propyl acetate, propylene carbonate, or propylene glycol.

In some embodiments, the solvent is ethyl acetate, butyl acetate, ethanol (denatured), isopropyl alcohol, acetone or mixtures and combinations thereof.

In some embodiments, the solvent is a ketone which is liquid at room temperature, such as methyl ethyl ketone, methyl isobutyl ketone, diisobutyl ketone, isophorone, cyclohexanone and acetone. In some embodiments, the solvent is an alcohol, such as ethanol, isopropanol, diacetone alcohol, 2-butoxyethanol and cyclohexanol. In some embodiments, the solvent is a glycol such as ethylene glycol, propylene glycol, pentylene glycol and glycerol. In some embodiments, the solvent is a propylene glycol ether which is liquid at room temperature, such as propylene glycol monomethyl ether, propylene glycol monomethyl ether acetate and dipropylene glycol mono-n-butyl ether. In some embodiments, the solvent is a short-chain ester (containing from 3 to 8 carbon atoms in total), such as ethyl acetate, methyl acetate, propyl acetate, n-butyl acetate and isopentyl acetate. In some embodiments, the solvent is an ether which is liquid at room temperature, such as diethyl ether, dimethyl ether and dichlorodiethyl ether. In some embodiments, the solvent is an alkane which is liquid at room temperature, such as decane, heptane, dodecane and cyclohexane. In some embodiments, the solvent is an aromatic cyclic compound which is liquid at room temperature, such as toluene and xylene. In some embodiments, the solvent is an aldehyde which is liquid at room temperature, such as benzaldehyde and acetaldehyde, and mixtures thereof.

### High-Molecular Weight (Meth)Acrylate Polymer or Copolymer

According to some embodiments, the nail coatings of the present embodiments may also include a high-molecular weight (meth)acrylate polymer or copolymer. While the compositions of the present embodiments can include acrylates, methacrylates are preferred because methacrylates are less likely to cause skin sensitization than acrylate formulas. The term ‘(meth)acrylate’ as used herein, means methacrylate, acrylate, or mixtures thereof.

In some embodiments, the high-molecular weight (meth)acrylate polymer or copolymer is a copolymer of an alkyl methacrylate (AMA) and methacrylic acid (MAA). The alkyl group may be, for example, methyl, ethyl, propyl or butyl. According to an aspect, the monomers are present in the polymer in a ratio of 90 parts AMA to 10 parts MAA (90:10 AMA/MAA). According to an aspect, the MAA monomer fraction may vary from 0 to 100% i.e. the (meth)acrylate polymer or copolymer may be an alkyl methacrylate polymer. According to an aspect, the AMA-MAA copolymer has a AMA:MAA monomer ratio of about 50:50. According to an aspect, the AMA-MAA copolymer has a AMA:MAA monomer ratio of about 60:40. According to an aspect, the AMA-MAA copolymer has a AMA:MAA monomer ratio of about 80:20. According to an aspect, the AMA-MAA copolymer has a AMA:MAA monomer ratio of about 90:10. According to an aspect, the AMA-MAA copolymer has a AMA:MAA monomer ratio of about 95:5.

In some embodiments, the high-molecular weight (meth)acrylate polymer or copolymer is a copolymer of methyl methacrylate (MMA) and methacrylic acid (MAA). According to an aspect, the monomers are present in the polymer in a ratio of 90 parts MMA to 10 parts MAA (90:10 MMA/MAA). According to an aspect, the MAA monomer fraction may vary from 0 to 100%; i.e. the (meth)acrylate polymer or copolymer may be a methyl methacrylate polymer. According to an aspect, the MMA-MAA copolymer has a MMA:MAA monomer ratio of about 50:50. According to an aspect, the MMA-MAA copolymer has a MMA:MAA monomer ratio of about 60:40. According to an aspect, the MMA-MAA copolymer has a MMA:MAA monomer ratio of about 80:20. According to an aspect, the MMA-MAA copolymer has a MMA:MAA monomer ratio of about 90:10. According to an aspect, the MMA-MAA copolymer has a MMA:MAA monomer ratio of about 95:5.

In some embodiments, the high-molecular weight (meth)acrylate copolymer is a copolymer of butyl methacrylate (BMA) and methacrylic acid (MAA). According to an aspect, the monomers are present in the polymer in a ratio of 90 parts BMA to 10 parts MAA (90:10 BMA/MAA). According to an aspect, the MAA monomer fraction may vary from 0 to 100% i.e. the (meth)acrylate polymer or copolymer may be a butyl methacrylate polymer. According to an aspect, the BMA-MAA copolymer has a BMA:MAA monomer ratio of about 50:50. According to an aspect, the BMA-MAA copolymer has a BMA:MAA monomer ratio of about 60:40. According to an aspect, the BMA-MAA copolymer has a BMA:MAA monomer ratio of about 80:20. According to an aspect, the BMA-MAA copolymer has a BMA:MAA monomer ratio of about 90:10. According to an aspect, the BMA-MAA copolymer has a BMA:MAA monomer ratio of about 95:5.

In some embodiments, the high-molecular weight (meth)acrylate polymer or copolymer has a molecular weight between 1,000 g/mol and 20,000 g/mol. In some embodiments, the high-molecular weight (meth)acrylate polymer or copolymer has a molecular weight of at least 2,000 g/mol. In some embodiments, the high-molecular weight (meth)acrylate polymer or copolymer has a molecular weight of at least 3,000 g/mol.

In some embodiments, the high-molecular weight (meth)acrylate polymer or copolymer has a molecular weight between 2,000 g/mol and 10,000 g/mol.

In some embodiments, the high-molecular weight (meth)acrylate polymer or copolymer has a molecular weight between 3,000 g/mol and 10,000 g/mol.

In some embodiments, the high-molecular weight (meth) polymer or copolymer has a molecular weight betwe

[symbol]

### Urethane (Meth)Acrylate Resin

According to some embodiments, the nail coatings of the present embodiments may also include a urethane (meth)acrylate resin. While the compositions of the present embodiments can include urethane acrylates, urethane methacrylates are preferred because urethane methacrylates are less likely to cause skin sensitization than acrylate formulas. The term ‘urethane (meth)acrylate’ as used herein, means urethane methacrylate, urethane acrylate, or mixtures thereof.

In some embodiments, the urethane (meth)acrylates have a molecular weight between 200 g/mol and 20,000 g/mol. In some embodiments, the urethane (meth)acrylates have a molecular weight of at least 2,000 g/mol. In some embodiments, urethane (meth)acrylates have a molecular weight of at least 3,000 g/mol. In some embodiments, the urethane (meth)acrylates have a molecular weight between 2,000 g/mol and 10,000 g/mol. In some embodiments, the urethane (meth)acrylates have a molecular weight between 3,000 g/mol and 10,000 g/mol.

In some embodiments, the urethane (meth)acrylate is an aliphatic polyol modified urethane methacrylate. Such molecules may be formed by reaction of reactants comprising an aliphatic polyol, a hydroxyalkyl methacrylate, and a diisocyanate, and having a weight average molecular weight (M_{W}) ranging from, for example, about 1000 to about 6000. Methods for making polyol modified urethane methacrylate without the use of diisocyanate are also known. In some embodiments, the aliphatic polyol is a polyether, polyester, polybutadiene, and/or polycarbonate.

For example, in some embodiments, the urethane (meth)acrylate is a an aliphatic polyesterpolyol based urethane methacrylate. Such molecules may be formed by reaction of reactants comprising an aliphatic polyesterpolyol, a hydroxyalkyl methacrylate, and a diisocyanate, and having a weight average molecular weight ranging from, for example, about 1000 to about 6000.

In some embodiments, the hydroxyalkyl methacrylate is selected from the group consisting of hydroxymethyl methacrylate, hydroxyethyl methacrylate, hydroxyproyl methacrylate, hydroxybutyl methacrylate, hydroxypentyl methacrylate, hydroxyhexyl methacrylate, and combinations thereof, and more preferably, the hydroxyalkyl methacrylate is hydroxyethyl methacrylate.

In some embodiments, the diisocyanate is selected from the group consisting of isophorone diisocyanate (IPDI), dicyclohexylmethane diiocyanate, 1-methylcyclohexane-2,4-diisocyanate, dicyclohexyl dimethyl-methane p,p′-diisocyanate, and combinations thereof. More preferably, the diisocyanate is isophorone diisocyanate.

In some embodiments, the urethane (meth)acrylate can be a polyester, polyether, polybutadiene and/or polycarbonate urethane oligomer (meth)acrylate.

In some embodiments, the urethane (meth)acrylate is a polyether urethane oligomer (meth)acrylate. By polyether urethane oligomer (meth)acrylate is meant a compound for example which contains at least polyether, urethane and (meth)acrylate groupings.

In some embodiments, the urethane (meth)acrylate is a polyester urethane oligomer (meth)acrylate. By polyester urethane oligomer (meth)acrylate is meant a compound, for example, which contains at least polyester, urethane and (meth)acrylate groups.

In some embodiments, the urethane (meth)acrylate is a polybutadiene urethane oligomer (meth)acrylate. By polybutadiene, urethane oligomer (meth)acrylate is meant a compound, for example, which contains at least polybutadiene, urethane and (meth)acrylate groups

In some embodiments, the urethane (meth)acrylate is a polycarbonate urethane oligomer (meth)acrylate. By polycarbonate, urethane oligomer (meth)acrylate is meant a compound, for example, which contains at least polycarbonate, urethane and (meth)acrylate groups.

These urethane oligomer (meth)acrylates are accessible, in that a polyester, polyether, polybutadiene and/or polycarbonate diol (diol component) with an aliphatic, cycloaliphatic and/or aromatic diisocyanate, for example 1,6-hexamethylene diisocyanate (HDI), 2,4,4-trimethylhexamethylene-1,6-diisocyanate (TMDI), tetramethylene diisocyanate, isophorone diisocyanate, 4,4′-dicyclohexylmethane diisocyanate, 1,4-phenylene diisocyanate, 2,6- and 2,4-toluene diisocyanate, 1,5-naphthylene diisocyanate, 2,4′- and 4,4′-diphenylmethane diisocyanate (diisocyanate component) are reacted under amine or tin catalysis. If a molar excess of diol component compared with diisocyanate component is hereby used, terminal OH groups remain which can be esterified with an ethylenically unsaturated acid such as acrylic acid or methacrylic acid or one of their derivatives. If a molar excess of diisocyanate component compared with diol component is used, terminal isocyanate groups remain which are reacted with a hydroxyalkyl and/or hydroxyaryl (meth)acrylate and/or di(meth)acrylate and/or tri(meth)acrylate, such as for example 2-hydroxyethyl acrylate (HEA), 2-hydroxyethyl methacrylate (HEMA), 3-hydroxypropyl methacrylate (HPMA), 3-hydroxypropyl acrylate (HPA), glycerol dimethacrylate and/or glycerol diacrylate.

Usable polycarbonate polyols are, for example, products which result from reaction with diols, such as 1,3-propanediol, 1,4-butanediol, 1,6-hexanediol, diethylene glycol, neopentyl glycol, trimethyl-1,6-hexanediol, 3-methyl-1,5-pentanediol and/or tetraethylene glycol, with diaryl carbonates such as diphenyl carbonate, or with phosgene.

Usable polyether polyols include for example products which are accessible by polymerization of a cyclic oxide, for example ethylene oxide, propylene oxide or tetrahydrofuran or by addition of one or more of these oxides to polyfunctional initiators such as water, ethylene glycol, propylene glycol, diethylene glycol, cyclohexane dimethanol, glycerol, trimethylol propane, pentaerythrite or Bisphenol A. Particularly suitable polyether polyols are polyoxypropylene diols and triols, poly(oxyethylene-oxypropylene) diols and triols which are obtained by simultaneous or sequential addition of ethylene and propylene oxide to suitable initiators, as well as polytetramethylene ether glycols, which result from polymerization of tetrahydrofuran.

In some embodiments, polyethers include polyethylene oxide, polypropylene oxide, polybutylene oxide.

In some embodiments, polyesters include polypropylene glycol, polyethylene glycol, polytetramethylene glycol, ethylene oxide-propylene oxide copolymer, tetrahydrofuran-ethylene oxide copolymer, tetrahydrofuran-propylene oxide copolymer, terephthalic acid, isophthalic acid, naphthalenedicarboxylic acid, cyclohexanedicarboxylic, 1,2-propane diol (propylene glycol), dipropylene glycol, diethylene glycol, 1,3-butanediol, ethylene glycol, and glycerol.

### Ethylenically Unsaturated Monomer

The ethylenically unsaturated monomer is, for example, methyl(meth)acrylate, ethyl(meth)acrylate, n-propyl(meth)acrylate, isopropyl(meth)acrylate, n-butyl(meth)acrylate, isobutyl(meth)acrylate, t-butyl(meth)acrylate, 2-ethylhexyl(meth)acrylate, cyclohexyl(meth)acrylate, stearyl(meth)acrylate, lauryl(meth)acrylate, trimethylcyclohexyl(meth)acrylate, isobornyl(meth)acrylate, or other alkyl(meth)acrylates; phenyl(meth)acrylate, benzyl(meth)acrylate, phenoxyethyl(meth)acrylate, phenoxydiethylene glycol (meth)acrylate, or other aromatic (meth)acrylates; tetrahydrofurfuryl(meth)acrylate, oxetane (meth)acrylate, or other heterocyclic (meth)acrylates; methoxy polypropylene glycol (meth)acrylate, ethoxy polypropylene glycol (meth)acrylate, or other alkoxy polyalkylene glycol (meth)acrylates; (meth)acrylamide, N,N-dimethyl(meth)acrylamide, N,N-diethyl(meth)acrylamide, N-isopropyl (meth)acrylamide, diacetone (meth)acrylamide, acryloylmorpholine, or other N-substituted (meth)acrylamides; N,N-dimethylaminoethyl(meth)acrylate, N,N-diethylaminoethyl(meth)acrylate, or other carboxyl-group-containing (meth)acrylates; or (meth)acrylonitrile, or other nitriles.

### Photoinitiator

Both UV and visible light activated photoinitiators may be suitable for the present invention. Suitable photoinitiator systems include aromatic alpha-hydroxy ketones, alkoxyoxybenzoins, acetophenones, acylphosphine oxides, bisacylphosphine oxides, and a tertiary amine plus a diketone, mixtures thereof and the like. Illustrative examples of photoinitiators are 1-hydroxycyclohexyl phenyl ketone, 2-hydroxy-2-methyl-1-phenyl-propan-1-one, bis(2,6-dimethoxybenzoyl)-2,4,4-trimethylpentyl phosphine oxide (DMBAPO), bis(2,4,6-trimethylbenzoyl)-phenyl phosphineoxide (Irgacure 819), 2,4,6-trimethylbenzyldiphenyl phosphine oxide and 2,4,6-trimethylbenzoyl diphenylphosphine oxide, benzoin methyl ester, camphorquinone, and a combination of camphorquinone and ethyl 4-(N,N-dimethylamino)benzoate. Commercially available visible light initiator systems include Irgacure 819, Irgacure 1700, Irgacure 1800, Irgacure 1850 (all from Ciba Specialty Chemicals) and Lucirin TPO initiator (available from BASF). Commercially available UV photoinitiators include Darocur 1173 and Darocur 2959 (Ciba Specialty Chemicals). The initiator is used in the reaction mixture in effective amounts to initiate photopolymerization of the reaction mixture, e.g., from about 0.01 to about 5 parts per 100 molar parts of reactive monomer. Alternatively, initiation can be conducted without a photoinitiator using, for example, e-beam. Preferred initiators include bisacylphosphine oxides, such as bis(2,4,6-trimethylbenzoyl)-phenyl phosphine oxide (Irgacure 819®) or a combination of 1-hydroxycyclohexyl phenyl ketone and bis(2,6-dimethoxybenzoyl)-2,4,4-trimethylpentyl phosphine oxide (DMBAPO), and the preferred method of polymerization initiation is visible light.

In some embodiments, visible light activated photoinitiators are preferred. The most preferred is bis(2,4,6-trimethylbenzoyl)-phenyl phosphine oxide (Irgacure 819®).

Viscosity of the coating may be controlled with the addition or removal of solvent.

### Methods

According to some embodiments, there is provided a method of applying a nail coating to an uncoated nail including the step of applying a nail coating according to any of the present embodiments. As used herein, the term “uncoated nail” may be a natural nail or an artificial nail. In some embodiments, a nail topcoat may be further applied to the coated nail surface, wherein the nail is coated with a nail coating according to the present embodiments. The method can also include the step of providing an uncoated nail. In some embodiments, the topcoat is a topcoat according to any of the embodiments described herein.

According to some embodiments, there is provided a method of applying a coating to a natural nail that includes the step of applying a nail coating according to any of the present embodiments to a natural nail. In some embodiments, a nail topcoat may be further applied to the coated nail surface, wherein the nail is coated with a nail coating according to the present embodiments. The method can also include the step of providing a natural nail. In some embodiments, the topcoat is a topcoat according to any of the embodiments described herein.

In some embodiments, the natural nail is not surface treated with a primer. In some embodiments, the natural nail is not surface treated with a file. In some embodiments, the natural nail is not roughened or otherwise texturized in order to promote the adhesion of a nail coating.

Methods of preparing polymer compositions by compounding nanostructured POSS chemicals into polymers are disclosed in U.S. Pat. No. 6,716,919, incorporated herein by reference in its entirety. Methods of dispersing particulates into a polymer by controlling its surface properties at the nanoscopic level with the use of nanostructured POSS chemicals are disclosed in U.S. Pat. No. 7,723,415, incorporated herein by reference in its entirety.

### DEFINITIONS

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting. Other features and advantages of the invention will be apparent from the following detailed description and claims.

For the purposes of promoting an understanding of the embodiments described herein, reference will be made to preferred embodiments and specific language will be used to describe the same. The terminology used herein is for the purpose of describing particular embodiments only, and is not intended to limit the scope of the present invention. As used throughout this disclosure, the singular forms “a,” “an,” and “the” include plural references unless the context clearly dictates otherwise. Thus, for example, a reference to “a composition” includes a plurality of such compositions, as well as a single composition.

### EXAMPLES

It is understood that modifications which do not substantially affect the activity of the various embodiments of this invention are also provided within the definition of the invention provided herein. Accordingly, the following examples are intended to illustrate but not limit the present invention. While the claimed invention has been described in detail and with reference to specific embodiments thereof, it will be apparent to one of ordinary skill in the art that various changes and modifications can be made to the claimed invention without departing from the spirit and scope thereof. Thus, for example, those skilled in the art will recognize, or be able to ascertain, using no more than routine experimentation, numerous equivalents to the specific substances and procedures described herein. Such equivalents are considered to be within the scope of this invention, and are covered by the following claims.

### Example 1

### Color Containing Base Formula

The base formula used to prepare exemplary color containing coatings according to the present embodiments is as follows:

| Component | Wt. % |
|---|---|
| Butyl Acetate | 43.361 |
| Ethyl Acetate | 28.582 |
| Nitrocellulose | 11.512 |
| Adipic Acid/Neopentyl Glycol/Trimellitic Anhydride Copolymer | 5.929 |
| Acetyl Tributyl Citrate | 4.941 |
| Isopropyl alcohol | 2.393 |
| Stealkonium bentonite | 1.207 |
| Red 7 Lake CI5850 | 0.506 |
| Red 6 Lake CI5850 | 0.488 |
| Alkyl Acrylate copolymer | 0.302 |
| Silica | 0.201 |
| TiO2 CI77891 | 0.182 |
| D&C black2 | 0.001 |
| Propylene carbonate | 0.198 |
| Benzophenone 1 | 0.198 |

### Example 2

### Nail Coatings and Testing

Exemplary color containing nail coatings according to the present embodiments were prepared and tested. Twenty-five different formulas were prepared using the ranges set forth in the table below:

| Component | Minimum | Maximum |
|---|---|---|
| EPO409 | 0.00% | 3.00% |
| Acetyl Tributyl Citrate | 0.00% | 2.50% |
| Ethyl Acetate* | 0.00% | 10.00% |
| Butyl Acetate* | 0.00% | 10.00% |
| Color Containing Base | 84.50% | 100.00% |
| Formula | | |

*Solvent constraint: The combination of Ethyl Acetate and Butyl Acetate ≦10%

One or more of the components of Ethyl Acetate, Butyl Acetate, Acetyl Tributyl Citrate, and EP0409 were added to the Color Containing Base Formula to prepare the test coatings of Formulas #1 to 25, provided in the table below. The amounts are provided in weight percent.

| | | | | | Color |
|---|---|---|---|---|---|
| | | Acetyl Tributyl | Ethyl | Butyl | Containing |
| Formula # | EP0409 | Citrate | Acetate | Acetate | Base Formula |
| 1 | 3 | 0 | 10 | 0 | 87 |
| 2 | 2 | 1.25 | 4.5 | 1.5 | 90.75 |
| 3 | 1 | 2.5 | 6 | 0 | 90.5 |
| 4 | 1 | 0 | 10 | 0 | 89 |
| 5 | 3 | 2.5 | 5 | 5 | 84.5 |
| 6 | 3 | 1.25 | 0 | 10 | 85.75 |
| 7 | 1 | 2.5 | 0 | 10 | 86.5 |
| 8 | 3 | 2.5 | 10 | 0 | 84.5 |
| 9 | 3 | 2.5 | 2 | 0 | 92.5 |
| 10 | 1 | 0 | 0 | 10 | 89 |
| 11 | 1 | 0 | 10 | 0 | 89 |
| 12 | 1 | 0 | 6 | 0 | 93 |
| 13 | 2 | 1.25 | 1.5 | 4.5 | 90.75 |
| 14 | 2.5 | 1.875 | 6.5 | 1.5 | 87.625 |
| 15 | 3 | 2.5 | 0 | 6 | 88.5 |
| 16 | 1 | 2.5 | 0 | 10 | 86.5 |
| 17 | 3 | 0 | 5 | 5 | 87 |
| 18 | 3 | 1.25 | 0 | 10 | 85.75 |
| 19 | 1 | 0 | 2 | 0 | 97 |
| 20 | 3 | 0 | 0 | 2 | 95 |
| 21 | 2 | 2.5 | 0 | 2 | 93.5 |
| 22 | 1 | 2.5 | 6 | 0 | 90.5 |
| 23 | 1 | 1.25 | 0 | 2 | 95.75 |
| 24 | 1 | 0 | 0 | 6 | 93 |
| 25 | 3 | 0 | 0 | 2 | 95 |
| Control | 0 | 0 | 0 | 0 | 100 |

The compositions of formulas I-25 were tested for adhesion, dry time and print resistance and compared to a control sample corresponding to a commercially available nail enamel. Dry time is a measure of how much time the composition required for drying.

Adhesion was measured using the Cross Hatch Adhesion test (ASTM D3359). Briefly, a crosshatch pattern is made though the film to the substrate. Detached flakes of coating are removed by brushing with a soft brush. Pressure-sensitive tape is applied over the crosshatch cut. Tape is smoothed into place by using a pencil eraser over the area of the incisions. Tape is removed by pulling it off rapidly back over itself as close to an angle of 180°. Adhesion is assessed on a 0 to 5 scale.

Print Resistance is a measure of a resistance of dried lacquer films to imprinting and this was tested using the Standard Test Method for Print Resistance of Lacquers (ASTM D 2091). Briefly, a weight presses a piece of fabric against the test surface. The surface is then examined and changes in appearance of the test surface are reported.

Table below summarizes the results from testing.

| | ADHESION | Dry Time | Print |
|---|---|---|---|
| FORMULA # | SCORE | (min) | Resistance |
| 21 | 4.21 | 13 | Medium Print |
| 5 | 4.04 | 12 | Medium Print |
| 15 | 4.00 | 14 | Medium Print |
| 6 | 3.96 | 10 | Slight-Medium |
| 7 | 3.88 | 12 | Medium Print |
| 8 | 3.88 | 8 | Medium Print |
| 14 | 3.83 | 13 | Medium Print |
| 3 | 3.58 | 13 | Medium Print |
| 2 | 3.50 | 10 | Slight Print |
| 11 | 3.50 | 10 | Slight Print |
| 16 | 3.50 | 8 | Medium Print |
| 1 | 3.42 | 8 | Medium Print |
| 18 | 3.42 | 15 | Medium Print |
| 17 | 3.33 | 11 | Medium Print |
| 9 | 3.33 | 13 | Medium Print |
| 10 | 3.33 | 12 | Slight Print |
| 13 | 3.33 | 14 | Slight-Medium |
| 20 | 3.25 | 11 | Medium Print |
| 25 | 3.25 | 2 | Medium Print |
| 4 | 3.17 | 10 | Slight Print |
| 22 | 3.17 | 13 | Slight-Medium |
| 19 | 2.83 | 12 | Medium Print |
| 24 | 2.83 | 11 | Slight-Medium |
| 12 | 2.75 | 6 | Slight Print |
| 23 | 2.67 | 11 | Medium Print |
| Control | 1.83 | 16 | Slight Print |

### Example 3

### Clear Base Formula

Two exemplary base formulas used to prepare exemplary clear coatings according to the present embodiments are as follows:

| | Wt. % | |
|---|---|---|
| | Clear Base | Clear Base |
| Component | Formula A | Formula B |
| Ethyl Acetate | 50.340 | 40.1000 |
| Butyl Acetate | 15.592 | 24.2499 |
| Phthalic Anhydride/Trimellitic Anhydride/Glycols | 11.508 | |
| Copolymer | | |
| Adipic Acid/Neopentyl Glycol/Trimellitic | | 7.2000 |
| Copolymer | | |
| Nitrocellulose | 11.389 | 13.0000 |
| Cellulose Acetate Butyrate | | |
| Acetyl Tributyl Citrate | 6.290 | 5.6000 |
| IPA | 4.881 | 5.6000 |
| Acrylates Copolymer | | |
| Heptane | | 4.0000 |
| Violet 2 CI60725 | 0.001 | 0.0001 |
| Benzophenone | | 0.2000 |
| Trimethylpentanediyl dibenzoate | | 0.0500 |

### Example 4

### Nail Coatings and Testing

Exemplary clear nail coatings according to the present embodiments were prepared, tested and compared to control samples. Control samples correspond to a commercially available clear nail enamel.

| | | Acetyl Tributyl | Clear Base | Clear Base |
|---|---|---|---|---|
| Formula # | EP0409 | Citrate | Formula A | Formula B |
| 1 | 2 | 2 | | 96 |
| 1 Control | 0 | 0 | | 100 |
| 2 | 2 | 2 | 96 | |
| 2 Control | 0 | 0 | 100 | |

The above compositions were tested for adhesion using the Cross Hatch Adhesion test (ASTM D3359). Briefly, a crosshatch pattern is made though the film to the substrate. Detached flakes of coating are removed by brushing with a soft brush. Pressure-sensitive tape is applied over the crosshatch cut. Tape is smoothed into place by using a pencil eraser over the area of the incisions. Tape is removed by pulling it off rapidly back over itself as close to an angle of 180°. Adhesion is assessed on a 0 to 5 scale.

Table below summarizes the results from testing.

| | | Improvement over |
|---|---|---|
| FORMULA # | ADHESION SCORE | Control |
| 1 | 3.0 | 71% |
| 1 Control | 1.75 | — |
| 2 | 2.25 | 125 |
| 2 Control | 1.0 | — |

### Example 5

### Nail Topcoat Base Formula

The base formula used to prepare exemplary topcoat formulations according to the present embodiments is as follows:

| Component | Wt. % |
|---|---|
| Ethyl Acetate | 40.1 |
| Butyl Acetate | 24.2 |
| Adipic Acid/Neopentyl Glycol/Trimellitic | 7.2 |
| Nitrocellulose or cellulose acetate butyrate | 13.0 |
| Acetyl Tributyl Citrate | 5.6 |
| Isopropyl alcohol | 5.6 |
| Heptane | 4.0 |
| Benzophenone | 0.2 |
| Trimethylpentanediyl dibenzoate | 0.1 |

### Example 6

### Nail Topcoats and Testing

Exemplary topcoat nail coating according to the present embodiments were prepared and tested. Thirty-eight different formulas were prepared using the ranges set forth in the table below:

| Component | Minimum | Maximum |
|---|---|---|
| EPO409 | 0.00% | 3.00% |
| Acetyl Tributyl Citrate | 0.00% | 3.00% |
| Isopropylidenediphenol PEG-2 | 0.00% | 5.00% |
| Dimethacrylate | | |
| Irgacure 819 (10% Solution) | 0.00% | 3.00% |
| Butyl Metacrylate/Methacrylic Acid | 0.00% | 7.50% |
| Copolymer 90/10 (33% in EtOH/Acetone) | | |
| Butyl Acetate | 0.00% | 7.50% |
| Nail Topcoat base formula | 75.00% | 100.00% |

One or more of the components of Butyl Methacrylate/Methacrylic Acid (BUMA) 90%/10% copolymer (33% in EtOH/Acetate), Urethane Methacrylate, Irgacure 819 (10% Solution), Butyl Acetate, Acetyl Tributyl Citrate, and EP0409 were added to the Topcoat Base Formula, provided above, to prepare the test coatings of Formulas #1 to 38, provided in the table below. The amounts are provided in weight percent.

| | | | | | | | Nail |
|---|---|---|---|---|---|---|---|
| | BUMA- | | Irgacure | | Acetyl | | Topcoat |
| | MMA | Urethane | 819 10% | Butyl | Tributyl | | Base |
| Formula # | copolymer | Methacrylate | Sol | Acetate | Citrate | EP0409 | Formula |
| 1 | 0.0 | 5.0 | 0.0 | 3.8 | 3.0 | 3.0 | 85.3 |
| 2 | 0.0 | 0.0 | 3.0 | 0.0 | 0.0 | 3.0 | 94.0 |
| 3 | 7.5 | 0.0 | 3.0 | 0.0 | 0.0 | 0.0 | 89.5 |
| 4 | 7.5 | 5.0 | 2.0 | 7.5 | 3.0 | 0.0 | 75.0 |
| 5 | 7.5 | 0.0 | 0.0 | 0.0 | 0.0 | 3.0 | 89.5 |
| 6 | 0.0 | 0.0 | 0.0 | 7.5 | 0.0 | 3.0 | 89.5 |
| 7 | 3.8 | 5.0 | 0.0 | 7.5 | 0.0 | 3.0 | 80.8 |
| 8 | 3.8 | 5.0 | 3.0 | 0.0 | 3.0 | 3.0 | 82.3 |
| 9 | 0.0 | 5.0 | 3.0 | 0.0 | 0.0 | 0.0 | 92.0 |
| 10 | 0.0 | 5.0 | 0.0 | 7.5 | 0.0 | 0.0 | 87.5 |
| 11 | 7.5 | 2.5 | 3.0 | 0.0 | 0.0 | 3.0 | 84.0 |
| 12 | 7.5 | 5.0 | 3.0 | 7.5 | 0.0 | 2.0 | 75.0 |
| 13 | 0.0 | 0.0 | 3.0 | 7.5 | 0.0 | 0.0 | 89.5 |
| 14 | 7.5 | 2.5 | 0.0 | 7.5 | 0.0 | 0.0 | 82.5 |
| 15 | 7.5 | 5.0 | 3.0 | 7.5 | 2.0 | 0.0 | 75.0 |
| 16 | 0.0 | 5.0 | 3.0 | 7.5 | 3.0 | 3.0 | 78.5 |
| 17 | 7.5 | 5.0 | 0.0 | 6.5 | 3.0 | 3.0 | 75.0 |
| 18 | 0.0 | 0.0 | 0.0 | 7.5 | 3.0 | 1.5 | 88.0 |
| 19 | 7.5 | 0.0 | 3.0 | 7.5 | 3.0 | 3.0 | 76.0 |
| 20 | 0.0 | 0.0 | 0.0 | 0.0 | 3.0 | 3.0 | 94.0 |
| 21 | 7.5 | 5.0 | 1.0 | 7.5 | 1.0 | 3.0 | 75.0 |
| 22 | 0.0 | 5.0 | 0.0 | 0.0 | 0.0 | 3.0 | 92.0 |
| 23 | 7.5 | 5.0 | 3.0 | 3.3 | 0.0 | 3.0 | 78.3 |
| 24 | 0.0 | 5.0 | 3.0 | 0.0 | 0.0 | 0.0 | 92.0 |
| 25 | 7.5 | 5.0 | 3.0 | 7.5 | 2.0 | 0.0 | 75.0 |
| 26 | 3.8 | 0.0 | 0.0 | 7.5 | 3.0 | 0.0 | 85.8 |
| 27 | 0.0 | 0.0 | 0.0 | 0.0 | 0.0 | 0.0 | 100.0 |
| 28 | 7.5 | 0.0 | 0.0 | 0.0 | 3.0 | 0.0 | 89.5 |
| 29 | 7.5 | 5.0 | 2.0 | 7.5 | 3.0 | 0.0 | 75.0 |
| 30 | 0.0 | 0.0 | 3.0 | 0.0 | 3.0 | 0.0 | 94.0 |
| 31 | 0.0 | 5.0 | 0.0 | 0.0 | 3.0 | 0.0 | 92.0 |
| 32 | 7.5 | 5.0 | 0.0 | 3.8 | 3.0 | 0.0 | 80.8 |
| 33 | 4.0 | 1.4 | 1.5 | 4.0 | 1.5 | 1.5 | 85.9 |
| 34 | 7.5 | 0.0 | 3.0 | 7.5 | 3.0 | 3.0 | 76.0 |
| 35 | 7.5 | 5.0 | 0.0 | 0.0 | 0.0 | 0.0 | 87.5 |
| 36 | 0.0 | 2.5 | 3.0 | 0.0 | 3.0 | 3.0 | 88.5 |
| 37 | 0.0 | 0.0 | 0.0 | 0.0 | 0.0 | 0.0 | 100.0 |
| (Control) | | | | | | | |
| 38 | 0.0 | 2.5 | 3.0 | 7.5 | 0.0 | 3.0 | 84.0 |

The compositions of formulas I-38 were tested for dry time, gloss and print resistance. Dry time is a measure of how much time the composition required for drying. Gloss was tested using a 20 degree gloss meter.

Pencil hardness measurements are used to determine the hardness of the coatings. The hardness of a coating, relative to a standard set of pencil leads, is determined by scratching the leads across the coating at a controlled angle of 45° for a distance of approximately ¼ inch (6.35 mm). The range of the pencil leads is from 6B (softest)-5B-4B-3B-2B-B-H-2H-3H-4H-5H-6H (hardest). The recorded rating indicates the hardness at which the pencil lead scratches the coating.

The Table below summarizes the results from testing.

| | Dry Time | | |
|---|---|---|---|
| Formula # | (min) | Gloss | Pencil Hardness rate |
| 3 | 6 | 171.3 | B |
| 27 | 7 | 190.4 | B |
| 28 | 7 | 186.6 | B |
| 35 | 7 | 186.4 | B |
| 37 (Control) | 7 | 185.1 | B |
| 10 | 8 | 187.0 | B |
| 12 | 8 | 188.4 | B |
| 14 | 8 | 181.6 | B |
| 19 | 8 | 183.4 | B |
| 24 | 8 | 187.4 | B |
| 33 | 8 | 189.7 | B |
| 5 | 9 | 168.6 | B |
| 11 | 9 | 188.1 | B |
| 25 | 9 | 190.2 | B |
| 26 | 9 | 185.8 | B |
| 29 | 9 | 188.4 | B |
| 32 | 9 | 184.7 | B |
| 34 | 9 | 186.2 | B |
| 2 | 10 | 167.1 | B |
| 20 | 10 | 189.3 | B |
| 21 | 10 | 184.3 | B |
| 23 | 10 | 183.0 | B |
| 30 | 10 | 182.9 | B |
| 9 | 11 | 176.0 | B |
| 13 | 11 | 188.6 | B |
| 15 | 11 | 186.2 | B |
| 38 | 11 | 185.3 | B |
| 4 | 13 | 171.8 | B |
| 6 | 13 | 167.0 | B |
| 18 | 13 | 185.6 | B |
| 22 | 13 | 184.9 | B |
| 31 | 13 | 184.4 | B |
| 7 | 14 | 188.5 | B |
| 17 | 14 | 189.5 | B |
| 1 | 16 | 165.9 | B |
| 8 | 16 | 167.8 | B |
| 16 | 16 | 176.3 | B |
| 36 | 16 | 183.3 | B |

What is claimed is:

1. A nail coating comprising:
at least one film-forming agent;
at least one silicone resin; and
solvent.

2. The nail coating of claim 1, wherein the at least one film-forming agent is a cellulosic resin.

3. The nail coating of claim 2, wherein the silicon resin and cellulosic resin are combined in a ratio of silicon resin to the cellulosic resin from about 1:10 to about 1:1.

4. The nail coating of claim 1, wherein the silicon resin is a polyhedral oligomeric silsesquioxane.

5. The nail coating of claim 2, wherein the cellulosic resin is nitrocellulose.

6. The nail coating of claim 2, wherein the cellulosic resin is a cellulose ester.

7. The nail coating of claim 2, wherein the cellulosic resin is a cellulose acetate alkylate.

8. The nail coating of claim 7, wherein the cellulosic resin is a cellulose acetate alkylate selected from the group consisting of cellulose acetate butyrate, cellulose acetate propionate, and mixtures thereof.

9. The nail coating of claim 1, further comprising at least one plasticizer.

10. The nail coating of claim 9, wherein the plasticizer is an alkyl citrate.

11. The nail coating of claim 9, wherein the plasticizer is acetyl tributyl citrate.

12. The nail coating of claim 9, wherein the plasticizer is an acetylated monoglyceride.

13. The nail coating of claim 1, further comprising at least one coloring agent.

14. A nail topcoat comprising:
at least one film-forming agent,
at least one silicone resin;
at least one photoinitiator;
solvent; and
a component selected from at least one ethylenically unsaturated monomer, at least one urethane (meth)acrylate or a combination thereof.

15. X. The topcoat of claim 14, wherein the silicon resin is a polyhedral oligomeric silsesquioxane.

16. The topcoat of claim 14, further comprising at least one plasticizer.

17. The nail coating of claim 16, wherein the plasticizer is an alkyl citrate.

18. The nail coating of claim 16, wherein the plasticizer is acetyl tributyl citrate.

19. The nail coating of claim 16, wherein the plasticizer is an acetylated monoglyceride.

20. The topcoat of claim 14, further comprising at least one high-molecular weight (meth)acrylate polymer or copolymer.

21. The topcoat of claim 20, wherein the (meth)acrylate polymer or copolymer is a copolymer of an alkyl (meth)acrylate and a (meth)acrylic acid.

22. The topcoat of claim 21, wherein the (meth)acrylate polymer or copolymer is a copolymer of an alkyl methacrylate and methacrylic acid.

23. The topcoat of claim 22, wherein the alkyl methacrylate is methyl methacrylate or butyl methacrylate.

24. The topcoat of claim 14, comprising at least one urethane (meth)acrylate resin.

25. The topcoat of claim 14, comprising at least one ethylenically unsaturated monomer.

26. The topcoat of claim 14, comprising at least one urethane (meth)acrylate resin and at least one ethylenically unsaturated monomer.

27. The topcoat of claim 14, comprising at least one urethane (meth)acrylate resin and further comprising at least one high-molecular weight (meth)acrylate polymer or copolymer.

28. The topcoat of claim 14, comprising at least one ethylenically unsaturated monomer and further comprising at least one high-molecular weight (meth)acrylate polymer or copolymer.

29. The topcoat of claim 14, comprising at least one urethane (meth)acrylate resin and at least one ethylenically unsaturated monomer and further comprising at least one high-molecular weight (meth)acrylate polymer or copolymer.

30. A nail coating system comprising the nail coating of claim 1 and a nail topcoat.

31. The system of claim 30, wherein the topcoat comprises:
at least one film-forming agent,
at least one silicone resin;
at least one photoinitiator;
solvent; and
a component selected from at least one ethylenically unsaturated monomer, at least one urethane (meth)acrylate or a combination thereof.

32. A multilayer nail coating system comprising at least a nail coating of claim 1 and a nail topcoat.

33. The system of claim 32, wherein the topcoat comprises:
at least one film-forming agent,
at least one silicone resin;
at least one photoinitiator;
solvent; and
a component selected from at least one ethylenically unsaturated monomer, at least one urethane (meth)acrylate or a combination thereof.

34. A method of applying a nail coating to an uncoated nail, the method comprising the step of:
applying the nail coating of claim 1 to the uncoated nail.

35. The method of claim 34, further comprising applying a nail topcoat to the coated nail surface.

36. A method of applying a coating to a natural nail comprising the step of:
applying a nail coating of claim 1 to the natural nail.

37. The method of claim 36, further comprising applying a nail topcoat to the coated nail surface.

38. The method of claim 36, wherein the natural nail is not surface treated with a primer.

39. The method of claim 36, wherein the natural nail is not surface treated with a file.

40. The method of claim 36, wherein the natural nail is not roughened or otherwise treated in order to promote the adhesion of a nail coating.
